(19) United States
(12) Patent Application Publication
(10) Pub. No.: US 2007/0298025 A1
(43) Pub. Date: Dec. 27, 2007

(54) Pharmaceutical Compositions and Methods for Reducing Body Fat

(75) Inventors: Itzik Harosh, Paris (FR); Benedicte Fournes, Bievres (FR); Sebastien Barradeau, Paris (FR)

Correspondence Address: MERCHANT & GOULD PC, P.O. BOX 2903, MINNEAPOLIS, MN, 55402-0903, US

(73) Assignee: Obe Therapy Biotechnology S.A.S., Evry (FR)

(21) Appl. No.: 11/748,880

(22) Filed: May 15, 2007

Related U.S. Application Data
(63) Continuation of application No. PCT/EP05/13020, filed on Nov. 15, 2005.
(60) Provisional application No. 60/659,399, filed on Mar. 9, 2005.
(60) Provisional application No. 60/627,164, filed on Nov. 15, 2004.

(51) Int. Cl. A61K 38/48; A61K 36/00; A61P 3/00

(52) U.S. Cl. 424/94.66; 424/758; 424/94.63

(57) ABSTRACT

The invention concerns a method of reducing body fat content of a subject in need thereof, the method comprising administering to the subject an agent capable of down-regulating activity and/or expression of at least one component participating in protein digestion and/or absorption. Such agents may be (i) an oligonucleotide directed to an endogenous nucleic acid sequence expressing said at least one component participating in said protein digestion and/or absorption or (ii) a protease inhibitor directed to said at least one component participating in protein digestion and/or absorption. The invention is particularly directed to a method of reducing body fat content of a subject in need thereof, the method comprising administering to the subject serine protease inhibitor inhibiting both enteropeptidase and trypsin activity.

This application is a continuation of International Application PCT/EP2005/013020 filed on Nov. 15, 2005, which claims priority to U.S. Provisional Application No. 60/627,164, filed Nov. 15, 2004, and U.S. Provisional Application 60/659,399 filed on Mar. 9, 2005 which applications are hereby incorporated by reference.

### FIELD AND BACKGROUND OF THE INVENTION

The present invention relates to pharmaceutical compositions and methods of reducing body fat.

Obesity is a multi-faceted chronic condition and is the most prevalent nutritional problem in the United States today. Obesity, a condition caused by an excess of energy intake as compared to energy expenditure, contributes to the pathogenesis of hypertension, type II or non-insulin dependent diabetes mellitus, hypercholesterolemia, hyperlipidemia, hypertriglyceridemia, heart disease, pancreatitis, and such common forms of cancer as breast cancer, prostate cancer, uterine cancer and colon cancer.

According to the 1999-2000 National Health and Nutrition Examination Survey (1999-2000 NAHNES, obesity and excessive weight affect more than 64% of the U.S. adult population and it is predicted that obesity will become the primary cause of mortality by 2005. This phenomenon is not limited to the U.S. as increased numbers of obese individuals have recently been reported in Europe.

Obesity related genes have previously been described in the art as targets for the treatment of obesity. The obese gene (ob), which encodes for the circulating hormone leptin, and the diabetes gene (db), which encodes for its receptor [Tartaglia et al., (1995) Cell 83(7): 1263-71; Zhang et al., (1994) Nature 372(6505): 425-32], have both received wide attention. Leptin appears to regulate adipose tissue mass and also to modulate eating behavior. Although studies have shown that subcutaneous therapy with recombinant leptin can produce weight loss in both obese and lean subjects, it was found that most obese patients have high levels of endogenous leptin and are therefore leptin-resistant, a phenomenon that resembles insulin-resistance in diabetic patients [Considine et al., (1996) N Engl J Med 334(5): 292-5; Maffei et al., (1996) Diabetes 45(5): 679-82]. Thus, obese patients are mostly rendered insensitive to leptin (endogenous or exogenous). Additional examples of obesity related genes include agouti (ag), tubby (tub), fat (fat), mahogany and neuropeptide Y (NPY) [Flier and Maratos-Flier (1998) Cell 92(4): 437-40; Spiegelman and Flier (1996) Cell 87(3): 377-89; Nagle et al., (1999) Nature 398: 148-152; Gunn et al., (1999) Nature, 398: 152-156], all of which are associated with satiety and appetite control by the central nervous system (CNS) and therefore have divergent physiological targets as well as affecting energy balance and obesity. In addition to these genes, it has been suggested that the mitochondrial uncoupling proteins (UCP) 1 and 2, by preventing ATP synthesis and thus increasing glucose utilization, may also serve as targets for obesity treatment [Fleury et al., (1997) Nat Genet. 15(3): 269-72; Boss et al., (1997) FEBS Lett 408: 39-42; Bouchard et al., (1997) Hum. Mol. Genet. 11: 1887-[889]. However, all these proposed targets, as well as other obesity related genes, are highly limited by both their non-specificity and their redundancy, leading to associated substantial side effects [Nagle et al., (1999) Nature 398: 148-152; Gunn et al., (1999) Nature, 398: 152-156; Lu et al., (1994) Nature 371: 799-802; Cool et al., (1997) Cell 88: 73-83]. Furthermore, a lean phenotype has never been observed as a result of a deficiency of any of these gene products. Based on the “thrifty genome” theory, (which is described in detail by Neel [Am. J. Hum. Genet. (1962), 14, 353-362] and Coleman [Science(1979) 263, 663-665]), it was proposed that in most cases the genetic component of obesity involves a complex network of many genes creating various redundant biochemical pathways that stimulate appetite or satiety. Alternative inherited pathways therefore compensate for the inhibition or activation of a single pathway in order to maintain the same energy equilibrium.

At present, only a limited number of drugs for treating obesity are commercially available. Unfortunately, while some of these drugs may bring short-term relief to the patient, a long-term successful treatment has not been achieved as yet. Exemplary methods of treating obesity are also disclosed in U.S. Pat. Nos. 3,867,539; 4,446,138; 4,588,724; 4,745,122; 5,019,594; 5,300,298; 5,403,851; 5,567,714; 5,573,774; 5,578,613 and 5,900,411.

One of the presently available drugs for treating obesity, developed by Hoffman-LaRoche, is an inhibitor of pancreatic lipase (PL). Pancreatic lipase is responsible for the degradation of triglycerides to monoglycerides. However, it has been associated with side-effects such as severe diarrhea resulting in absorption inhibition of only one specific fraction of fatty acids and, has been known to induce allergic reactions. Treatment with PL inhibitors is thus highly disadvantageous and may even expose the treated subject to life-threatening risks.

Recently, it has been suggested that fat absorption may be reduced by inhibiting the activity of the microsomal triglyceride-transfer protein (MTP), which is involved in the formation and secretion of very light density lipoproteins (VLDL) and chylomicrons. Sharp et al., [Nature (1993) 365:65-69] and Wetterau et al., [Science (1994) 282:751-754,] demonstrated that the mtp gene is responsible for abetalipoproteinemia disease. U.S. Pat. Nos. 6,066,650, 6,121,283 and 6,369,075 describe compositions that include MTP inhibitors, which are aimed at treating various conditions associated with excessive fat absorption. However, patients treated with MTP inhibitors suffer major side effects including hepatic steatosis, which are attributed to reduced MTP activity in both intestine and liver. This is not surprising since people naturally deficient for MTP activity were shown to develop fatty livers [Kane and Havel (1989); Disorders of the biogenesis and secretion of lipoproteins containing the apolipoprotein B. pp. 1139-1164 in: “The metabolic basis of inherited disease” (Scrivers et al., eds.). McGraw-Hill, New York]. In fact, the company Brystol Myers Squibb, that developed MTP inhibitors for the treatment of obesity, has recently decided to abandon this target, due to this fatty liver side effect.

The presently known targets for the treatment of obesity and related disorders can be divided into four main classes: (i) appetite blockers, which include for example the NPY neuropeptide; (ii) satiety stimulators, which include, for example, the product of the ob, db and agouti genes; (iii) energy or fatty acid burning agents, which include the UCPs; and (iv) fat absorption inhibitors such as those acting on PL and MTP in the intestine, described above.

As is discussed hereinabove, the use of these targets is highly limited by their redundancy, their multiple targeting and/or their lack of tissue specificity.

There is thus a widely recognized need for, and it would be highly advantageous to have compositions and methods for treating obesity and related diseases and disorders devoid of the above limitations.

Energy is provided by carbohydrates (providing 25% of the energy), fat (providing 50% of the energy) and proteins (providing 25% of the energy).

Protein metabolism strikes a balance between the body's energy and the synthetic needs and may contribute to the development of obesity. The four major components of protein metabolism include protein synthesis, protein degradation, oxidation of amino acids and dietary intake of amino acids. When the dietary intake of protein is satisfactory, there is equilibrium between the various components of protein metabolism. Essentially, protein synthesis equilibrates with protein degradation. However, in many industrialized countries such as the United States, protein intake largely exceeds the needs of the individual. Thus, following mealtime, amino acid accumulation together with increased insulin, stimulates the storage of amino acids as protein. When the anabolic pathway is saturated, excess amino acids are oxidized. Oxidation products may either be used as substrates for energy production or may be converted to fat and stored in adipocytes, resulting in weight gain and ultimately contributing to the development of obesity.

On the other end of the scale, in times of starvation when glucose is scarce, gluconeogenesis occurs. Very little gluconeogenesis occurs in the brain, skeletal and heart muscles or other body tissues even though these organs have a high demand for glucose. Therefore, gluconeogenesis is constantly occurring in the liver to maintain the glucose level in the blood to meet these demands. However, in times of starvation, proteolytic degradation also plays a role in gluconeogenesis. Muscle releases lactate and glucogenic amino acids, that are converted to glucose in the liver via gluconeogenesis by direct entry into the citric acid cycle.

Protein metabolism provides 25% of food energy. Excess dietary amino acids are oxidized and the end-products are used either to produce energy or converted to fat. The present inventors postulated that limiting dietary amino acid absorption (by inhibiting proteolytic degradation of proteins) can be used to treat obesity, since limiting amino acid absorption would ultimately result in reduction of body fat formation.

According to the “thrifty genome” theory, obesity genes may have conferred, in times of shortage of nutrition, some evolutionary advantages through efficient energy exploitation. Nevertheless, when food is abundant and way of life become sedentary, the same genes yield to obesity, type II diabetes and other obesity-related diseases. It is a challenge to identify crucial gene(s) in which mutations result in reduced energy intake. However “expenditure genes” or “lean genes” (as opposed to obesity genes) can also be considered as new potential targets for the treatment of obesity. These genes can be identified in rare genetic diseases with lean, failure to thrive, malnutrition and/or energy malabsorption phenotype. For example, the congenital enteropeptidase deficiency, caused by mutations in the gene encoding the proenteropeptidase is characterized by a low body mass [A. Holzinger et al.; Am. J. Hum. Genet, 70:20-25; (2002)]. This pathology is usually successfully treated by pancreatic enzyme replacement or by dietary protein hydrolysate [Polonovski C, (1970). Arch. Franc. Péd 27:677-688]. A close pathology, the hydrochloric acid deficiency or achlorhydria, is also characterized by protein malabsorption and by a failure to thrive. In this pathology, the gastric pH is not acidic enough (above four). Pepsins are therefore not activated, and consequently ingested proteins are not digested into peptides. This ultimately leads to a considerably reduced intestinal digestion output.

Based on these observations correlating the EP deficiency or inactive pepsins with a thin phenotype and while searching for a novel therapeutic modality for obesity and related diseases, which would be devoid of the severe side effects known with the actually existing drugs, the present inventors postulated that pepsin activity, EP activity and/or underlying dietary enzymes activated thereby, may serve as selective and efficient targets for treating obesity.

### SUMMARY OF THE INVENTION

It is an object of the present invention to provide methods for reducing body fat of a subject.

It is another object of the present invention to provide compositions for treating a condition or disorder in which reducing body fat content is beneficial.

It is yet another object of the present invention to provide methods of treating a disease for which a low protein diet is beneficial in a subject.

Hence, according to the present invention there is provided a method of reducing body fat content of a subject in need thereof, the method comprising administering to the subject a therapeutically effective amount of an agent capable of down-regulating activity and/or expression of at least one component participating in protein digestion and/or absorption, thereby reducing the body fat content of the subject.

According to another aspect of the present invention there is provided a method of reducing body fat content of a subject in need thereof, the method comprising administering to the subject a therapeutically effective amount of an agent capable of down-regulating activity and/or expression of at least one component of an enteropeptidase pathway, thereby reducing the body fat content of the subject.

According to yet another aspect of the present invention there is provided a method of reducing a body fat content of a subject in need thereof, the method comprising administering to the subject a therapeutically effective amount of an agent capable of down-regulating activity and/or expression of pepsin, thereby reducing the body fat content of the subject.

According to still another aspect of the present invention there is provided a pharmaceutical composition for treating a condition or disorder in which reducing body fat content is beneficial, comprising, as an active ingredient, a therapeutic effective amount of an agent capable of down-regulating activity and/or expression of at least one component participating in protein digestion and/or absorption and a pharmaceutically acceptable carrier.

According to an additional aspect of the present invention there is provided an article of manufacture comprising packaging material and a pharmaceutical composition identified for reducing body fat content of a subject in need thereof being contained within the packaging material, the pharmaceutical composition including as an active ingredient an agent capable of down-regulating activity and/or expression of at least one component participating in protein digestion and/or absorption pathway and a pharmaceutically acceptable carrier.

According to yet an additional aspect of the present invention there is provided a method of treating a disease for which low protein diet is beneficial in a subject in need thereof, the method comprising providing to the subject a therapeutically effective amount of an agent capable of down-regulating activity and/or expression of at least one component participating in protein digestion and/or absorption, thereby treating the disease for which low protein diet is beneficial in the subject in need thereof.

According to further features in preferred embodiments of the invention described below, the component participating in protein digestion and/or absorption is a protease, particularly a serine-protease or an aspartate-protease.

According to still further features in the described preferred embodiments, the protease is at least one component of an enteropeptidase pathway.

According to still further features in the described preferred embodiments, the at least one component of an enteropeptidase pathway is an activator of enteropeptidase.

According to still further features in the described preferred embodiments, the activator of enteropeptidase is duodenase.

According to still further features in the described preferred embodiments, the at least one component of an enteropeptidase pathway is enteropeptidase.

According to still further features in the described preferred embodiments, the at least one component of an enteropeptidase pathway is a downstream effector of enteropeptidase.

According to still further features in the described preferred embodiments, the downstream effector of enteropeptidase is selected from the group consisting of trypsin, chemotrypsin, elastase, carboxypeptidase A, carboxypeptidase B and pancreatic lipase.

According to still further features in the described preferred embodiments, the protease is a pepsin.

According to still further features in the described preferred embodiments, the pepsin is selected from the group consisting of Pepsin A, Pepsin B and Gastricin.

According to still further features in the described preferred embodiments, down-regulating activity and/or expression of at least one component participating in protein digestion and/or absorption is effected by an agent selected from the group consisting of: (i) an oligonucleotide directed to an endogenous nucleic acid sequence expressing at least one component participating in protein digestion and/or absorption; (ii) a protease inhibitor directed to at least one component participating in protein digestion and/or absorption.

According to still further features in the described preferred embodiments, the protease inhibitor is an aspartic protease inhibitor.

According to still further features in the described preferred embodiments, the aspartic protease inhibitor is a peptidomimetic aspartic protease inhibitor.

According to still further features in the described preferred embodiments, the peptidomimetic aspartic protease inhibitor is selected from the group consisting of CGP53437, Amprenavir, Atazanavir, Indinavir, Lopinavir, Fosamprenavir, Nelfinavir, Ritonavir and Saquinavir.

According to still further features in the described preferred embodiments, the aspartic protease inhibitor is a low molecular weight aspartic protease inhibitor.

According to still further features in the described preferred embodiments, the low molecular weight aspartic protease inhibitor is pepstatin.

According to still further features in the described preferred embodiments, the aspartic protease inhibitor is extracted from a plant.

According to still further features in the described preferred embodiments, the plant is selected from the group consisting of Solanum tuberosum (potato), Cucurbita maxima (squash) and Anchusa strigosa (Prickly Alkanet).

According to still further features in the described preferred embodiments, the aspartic protease inhibitor is extracted from a parasite.

According to still further features in the described preferred embodiments, the parasite is selected from the group consisting of Ascaris suum and Ascaris lombricoides.

According to still further features in the described preferred embodiments, the aspartic protease inhibitor is pepsine inhibitor-3 (PI-3).

According to still further features in the described preferred embodiments, the protease inhibitor is a serine protease inhibitor.

According to still further features in the described preferred embodiments, the serine protease inhibitor is a low molecular weight serine protease inhibitor.

According to still further features in the described preferred embodiments, the serine protease inhibitor is a peptidomimetic serine protease inhibitor.

According to still further features in the described preferred embodiments, the agent is linked to a mucoadhesive agent.

According to still further features in the described preferred embodiments, the mucoadhesive agent is a mucoadhesive polymer.

According to still further features in the described preferred embodiments, the mucoadhesive polymer is selected from the group consisting of chitosan, polyacrylic acid, hydroxyprpyl methylcellulose and hyaluronic acid.

According to still further features in the described preferred embodiments, the subject in need thereof is afflicted with a condition or disorder selected from the group consisting of excessive weight, obesity, type II diabetes, hypercholesterolemia, atherosclerosis, hypertension, pancreatitis, hypertriglyceridemia and hyperlipidemia.

According to still further features in the described preferred embodiments, the administering to the subject is effected by oral administration.

The present invention successfully addresses the shortcomings of the presently known configurations by providing methods of reducing body fat content.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more details than is necessary for a fundamental understanding of the invention, the description taken with the drawings making apparent to those skilled in the art how the several forms of the invention may be embodied in practice.

In the drawings:

FIG. 1 is a scheme illustrating components of the initial pepsin digestion of dietary proteins (right) and of the enteropeptidase activation cascade (left).

FIG. 2 is the nucleic sequence and corresponding amino acid sequence of the human enteropeptidase (PRSS7)

The first line indicates the nucleotide sequence, grouped by codons; the second line indicates the amino acid sequence corresponding to the above codons with the three-letter code. The first codon of translation is shown in bold as well as the stop codon. Numbering of the nucleic acids is at the right end of the first line, whereas numbering of the amino acids is indicated under amino acid residue (third line).

FIG. 3 is the nucleic sequence and corresponding amino acid sequence of the human trypsin (PRSS1)

The first line indicates the nucleotide sequence, grouped by codons; the second line indicates the amino acid sequence corresponding to the above codons with the three-letter code. The first codon of translation is shown in bold as well as the stop codon. Numbering of the nucleic acids is at the right end of the first line, whereas numbering of the amino acids is indicated under amino acid residue (third line).

FIG. 4 is the acidic propeptide of tryspinogen. The vertical arrow shows the site of cleavage of the tryspsinogen by the enteropeptidase, between the Lys (P1) and the Ile, releasing the activation peptide (left part) and the active form of trypsin (right part).

FIG. 5 is a scheme of the trypsinogen activation assay. The release of pNA (p-nitroaniline) is measured as the result of the successful cleavage of the substrate N-CBZ-Gly-Pro-Arg-pNA by trypsin, which activity is the result of the cleavage of the tryspinogen by enteropeptidase.

FIG. 6 is IC50 measurements calculated by the trypsinogen activation assay. The graphs represent the percentage of inhibition (as compared to a value without inhibitor) in function of various concentrations of inhibitors, i.e. AC-Leu-Val-Lys-Aldhehyde (A), H-D-Tyr-Pro-Arg-chloromethylketone trifluororoacetate salt (B) and Z-Asp-Glu-Val-Asp-chloromethylketone (C).

### DESCRIPTION OF THE PREFERRED EMBODIMENTS

The present invention is of pharmaceutical compositions and methods of reducing body fat content.

The principles and operation of the present invention may be better understood with reference to the drawings and accompanying descriptions.

Before explaining at least one embodiment of the invention in details, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Excessive weight and obesity are widely recognized health problems, with approximately 97 million people considered clinically overweight or obese in the United States alone. These two conditions are associated with a number of psychological and medical ailments including atherosclerosis, hypertension, type II or non-insulin dependent diabetes mellitus, pancreatitis, hypercholesterolemia and hyperlipidemia.

Obesity results from greater energy intake than energy expenditure. Thus, treatment of obesity seeks to re-address this balance so that energy input is reduced below energy expenditure.

While conceiving the present invention, the inventors postulated that limiting protein digestion and/or absorption can be used as a method for reducing body fat content, and as such for treating obesity and related diseases.

Energy is provided by the ingestion of carbohydrates (providing 25% of the energy), fat (providing 50% of the energy) and proteins (providing 25% of the energy).

Glucose is the metabolite of choice of both brain and working muscle. It cannot be synthesized from fatty acids because neither pyruvate nor oxaloacetate, the precursors of glucose in gluconeogenesis, can be synthesized from acetyl-CoA. During starvation, glucose must therefore be synthesized from amino acids derived from the proteolytic degradation of proteins, the major source of which is muscle, resulting in loss of muscular mass.

Protein metabolism strikes a balance between the body's energy and the synthetic needs and contributes to the development of obesity. The four major components of protein metabolism are protein synthesis, protein degradation, amino acid oxidation and dietary intake of amino acids. When the dietary intake of protein is satisfactory, there is an equilibrium between the various components of protein metabolism. Essentially, protein synthesis equilibrates with protein degradation. However, in many industrialized countries such as the United States, protein intake largely exceeds the needs of the individual. Thus, following mealtime, amino acid intake together with increased insulin, stimulates the storage of amino acids as protein. When the anabolic pathway is saturated, excess amino acids are oxidized. The subsequent oxidation products are either used to produce energy or are converted to fat and stored in adipocytes, resulting in weight gain and ultimately contributing to the development of obesity.

Therefore, the limiting of excess amino acid absorption by the inhibition of protein degrading enzymes should assist in the prevention of body fat accumulation. Furthermore, it is believed that limiting excess amino acid absorption does not prohibit the body from metabolizing the continued supplies of fat and carbohydrates. However, since these sources are insufficient to compensate for the energy loss resulting from poor amino acid absorption, depletion in fat and carbohydrate (i.e. glycogen) stores should occur [Guyton and Hall “The Textbook of Medical Physiology” 10^{th} Ed. Harcourt International Edition].

Thus, the present invention can be successfully used for reducing body fat content in an individual. As an illustration, an individual consuming 2000 kcal/day and burning 1800 kcal will have an excess 200 kcal which, in turn, will be transformed to fatty acids and stored as fat, thereby gaining weight. Treating such an individual with agents of the present invention (further described herein below) at a concentration which would limit protein digestion and/or absorption and reduce the number of calories assimilated to less than those expended would enable weight loss. Since 25% of energy is attributed to protein metabolism a maximum number of 500 calories can be prevented from being assimilated by the present invention. However, since there is an amount of protein and corresponding amino acids that is essential to the body, measures are taken such that only a proportion of proteins is not digested and/or absorbed and thus not the total 500 calories should be prevented from being assimilated but a proportion thereof, as further described hereinbelow.

Thus, according to one aspect of the present invention there is provided a method of reducing body fat content of an individual subject.

As used herein the phrase “reducing body fat content” refers to reducing levels of mobilizable fat (e.g., fat contained in the blood) and fat tissue, which contains stored fat (e.g., adipose tissue).

As used herein the term “fat” refers to glycerol esters of saturated fatty acids such as triglycerides and fat-like substances such as steroid alcohols such as cholesterol.

The method, according to this aspect of the present invention is effected by providing to a subject in need thereof (e.g., an obese individual) a therapeutically effective amount of an agent capable of down-regulating activity and/or expression of at least one component participating in protein digestion and/or absorption, thereby limiting body fat storage and, therefore enhancing fat catabolism in fat cells of the subject thereby reducing the body fat mass of the subject.

The phrase “fat catabolism” refers to the process of breaking down ingested and stocked fat into fatty acids and glycerol and subsequently into simpler compounds that can be used by the body as a source of energy.

As used herein, the phrase “subject in need thereof” refers to a mammal, preferably a human, which can benefit from enhancing its fat catabolism using the agents of the present invention. Examples are human subjects or domestic animals (e.g., cats, dogs, cattle, sheep, pigs, goats, poultry and equines) that suffer from the diseases or conditions listed hereinbelow.

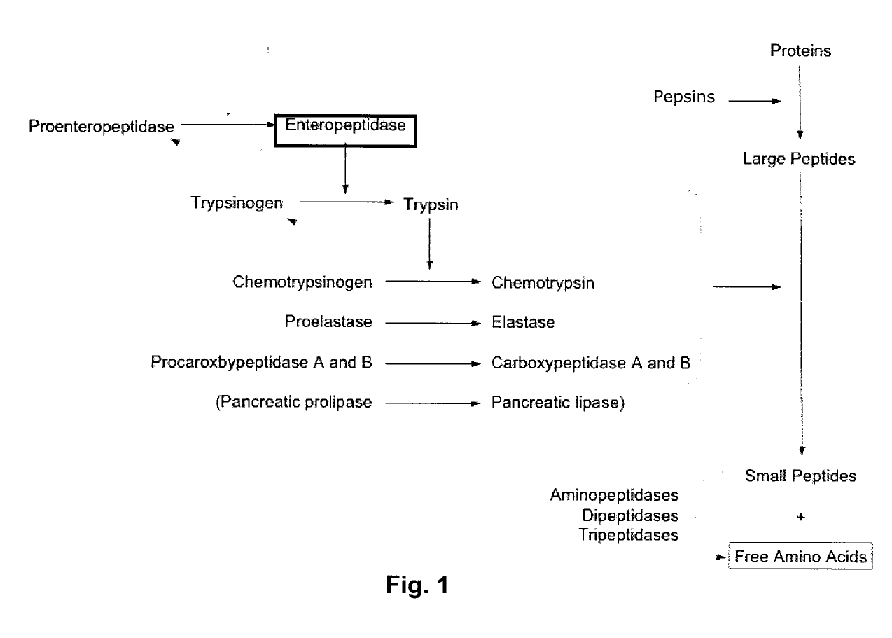

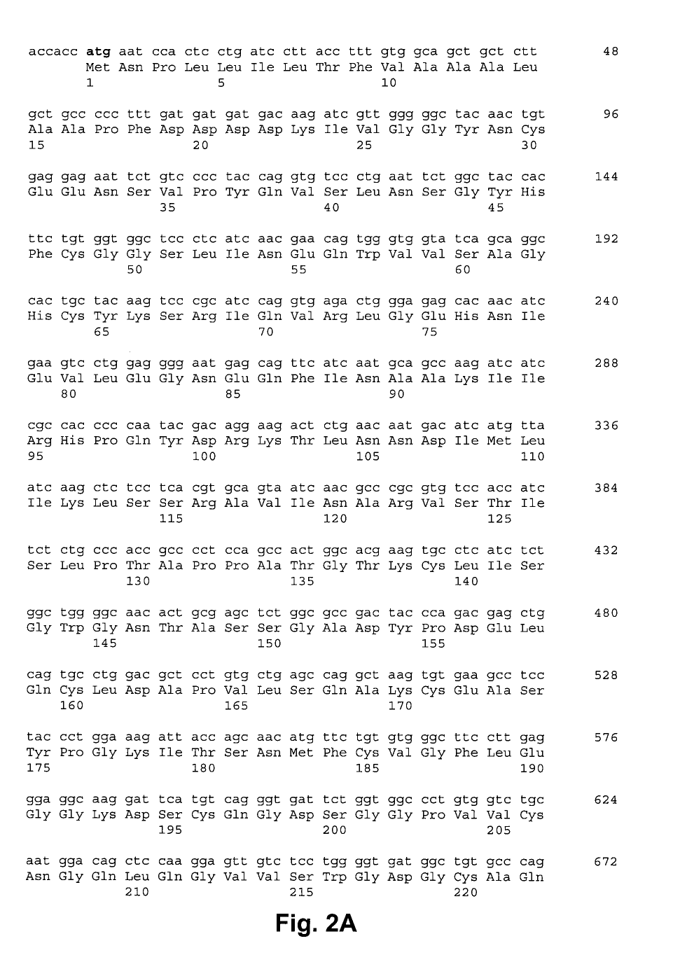

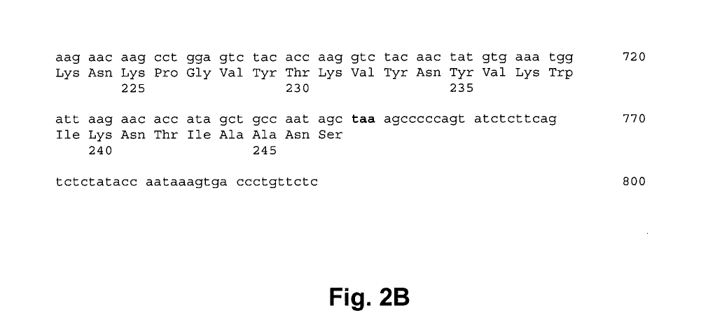

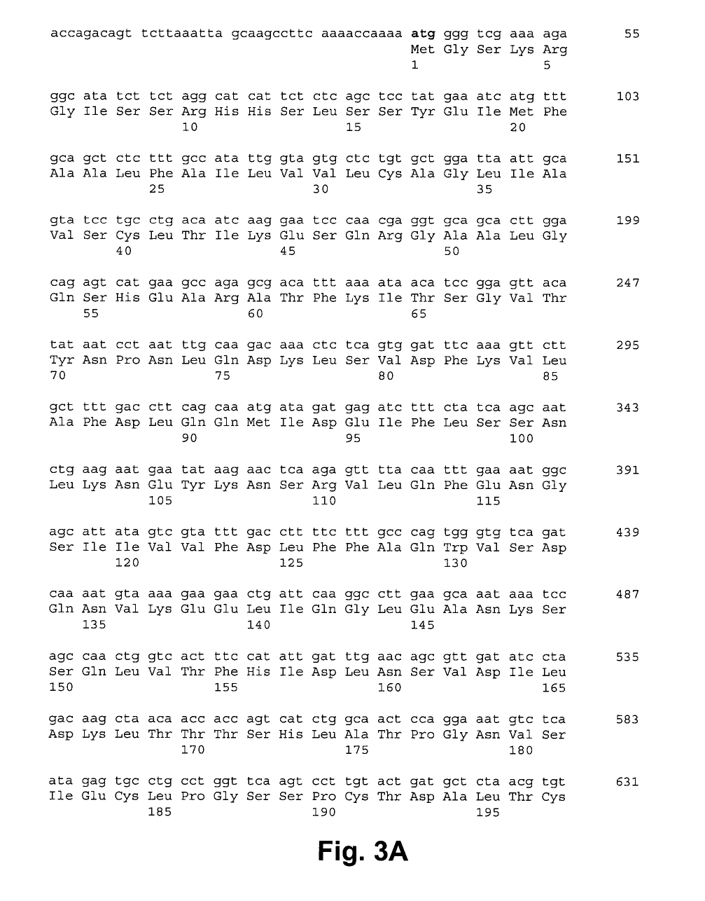

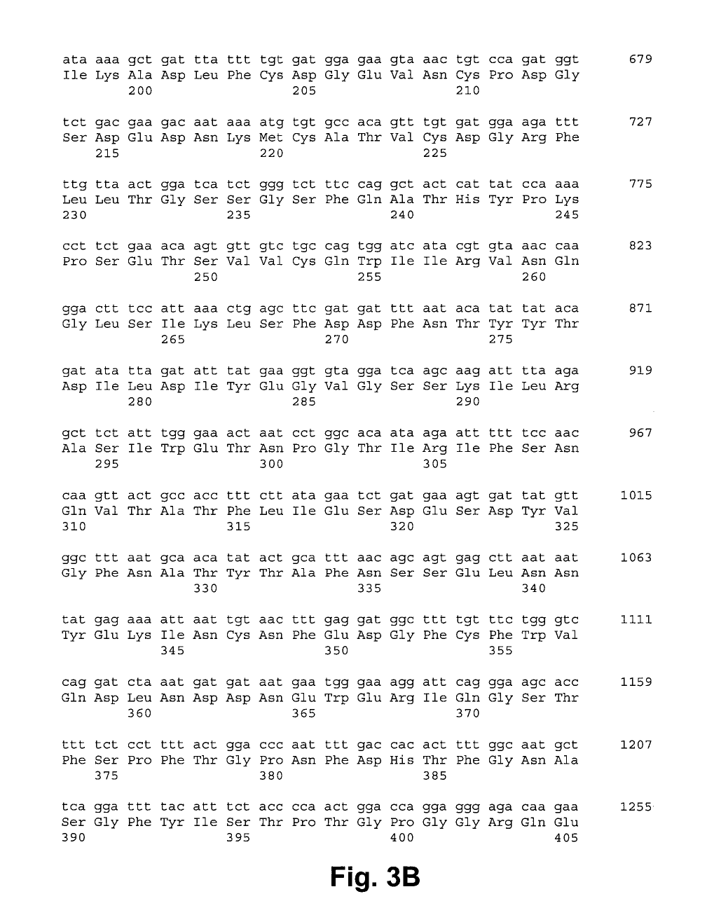

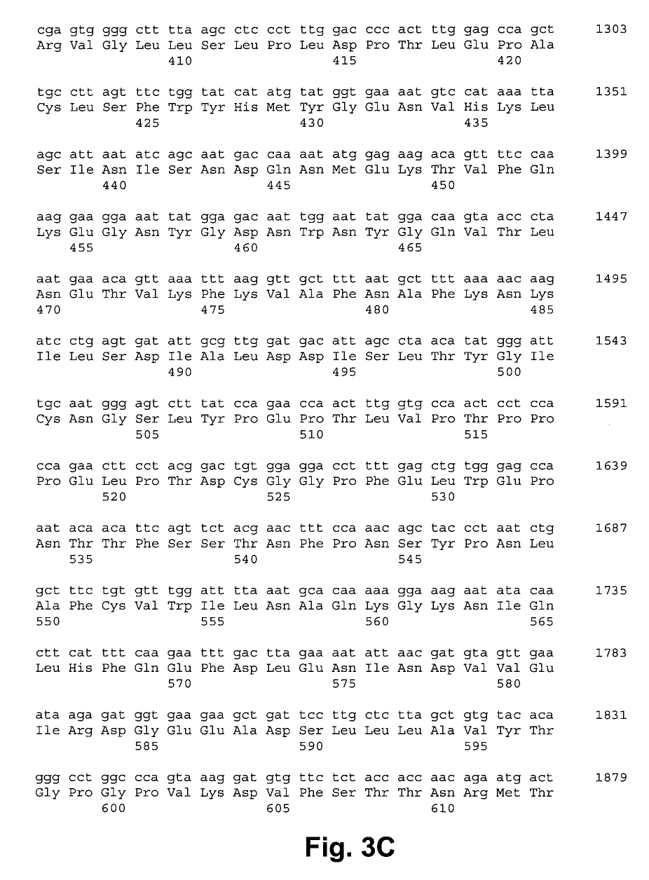

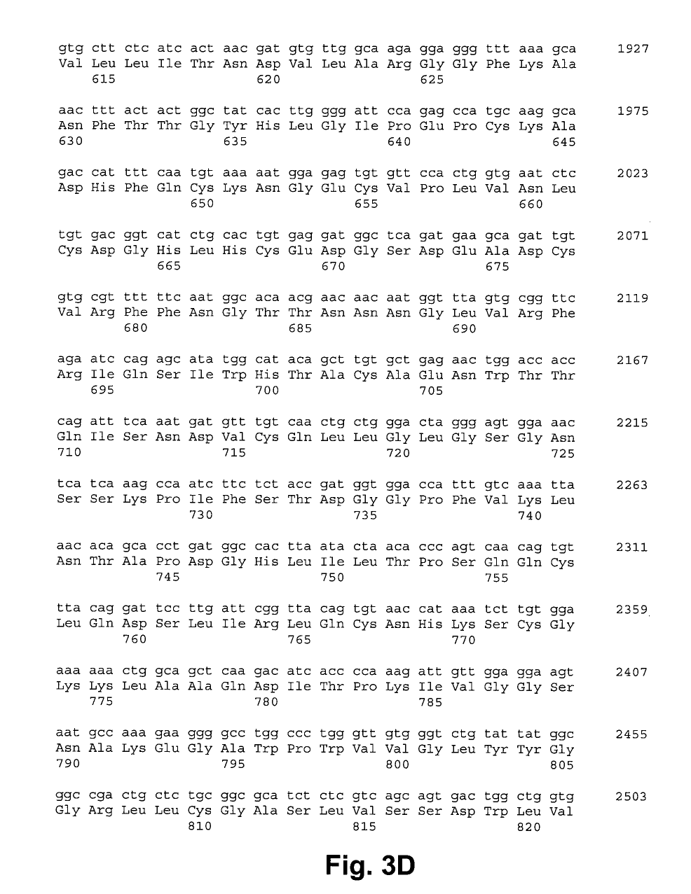

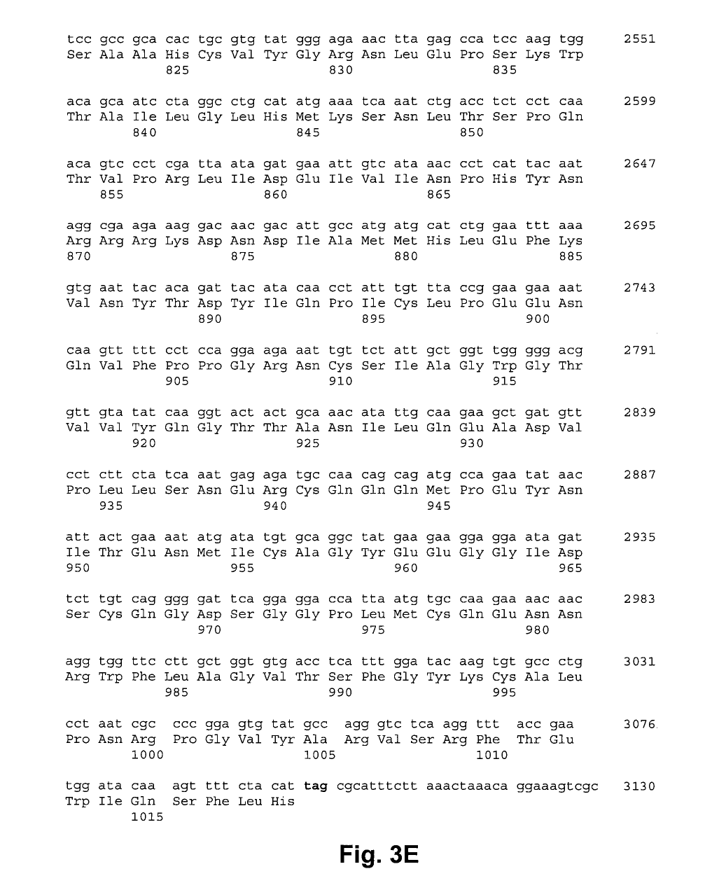

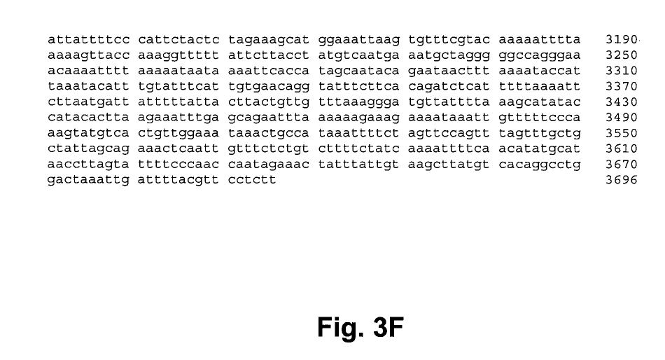

As used herein, the phrase “protein digestion” refers to the process by which proteins are broken down into peptides and amino acids. This process is effected in both the stomach and the small intestine (FIG. 1). Components which participate in protein digestion include proteolytic enzymes (i.e., proteases) and agents thereof including co-factors which are responsible for their activation.

As used herein, the phrase “protein absorption” refers to the process of amino acid and peptide absorption. This process is effected in the small intestine. Components, which participate in amino acid absorption, include amino acid receptors and transporters (e.g., sodium dependent amino acid transporters).

Preferably, in a first embodiment, the method of the invention is effected by down-regulating the expression and/or the activity of a protease that participates in protein digestion and/or absorption.

As used herein a “protease” refers to an enzyme that cleaves peptide bonds, which link amino acids together in protein molecules. Proteases comprise two groups of enzymes: (1) the endopeptidases that cleave peptide bonds within the protein and (2) the exopeptidases, which cleave peptide bonds removing amino acids sequentially from either the N or the C-terminus, respectively.

Preferably, in a first embodiment, the method of the present invention is effected by down-regulating the stomach enzyme, pepsin, which is active in the first step of protein digestion, breaking down proteins into large peptides. Pepsin is the active form of its inactive precursor pepsinogen (i.e., zymogen) where the acid environment of the stomach triggers its activation. Protein chains bind in the deep active site groove of pepsin, and are degraded into large peptides, which are later degraded into small peptides by intestinal enzymes. It is suggested that blockade of the first step of protein digestion would reduce further protein absorption in the intestine. Noteworthy, hydrochloric acid deficiency or achlorhydria, is characterized by protein malabsorption and by a failure to thrive. In this pathology, the gastric pH is not acidic enough (above four) to convert pepsinogen to pepsin. Consequently ingested proteins are not digested into peptides. This ultimately leads to a considerably reduced intestinal digestion output.

The pepsin family has three members, Pepsin A, Pepsin B and Gastricin, all of which belong to the aspartic protease family. They are all expressed in the stomach and are the first proteolytic enzymes of the gastrointestinal digestive system [See FIG. 1]. These enzymes are responsible for the break-down of proteins into large peptides. As these three enzymes are very similar, they are usually referred to indistinctly as Pepsins.

The aspartic protease family exists in vertebrates, plants and viruses. It includes Pepsins, the Cathepsin D, the Angiotesin-Converting Enzyme, the β-secretase and the HIV protease. They are characterized by the highly conserved sequence of Asp-Thr-Gly and are, with the exception of HIV protease which is a dimer of two identical subunits, monomeric enzymes comprising two domains. In general, aspartic proteases are highly specific cleaving peptide bonds between hydrophobic residues as well as a beta-methylene group. Pepsins, however, are considered to be proteases with broad structural specificity; an essential characteristic for their role in digestion. They do however elicit a preference for aromatic amino acid residues like phenylalanine. As used herein, pepsin refers to an aspartic protease of the pepsin family [e.g., Pepsin A (e.g., EC 3.4.23.1), Pepsin B (e.g., EC 3.4.23.2) and Gastricin eg. (EC 3.4.23.3) and to zymogens thereof such as, for example, Pepsinogen A (e.g., EC 3.4.23.1) Pepsinogen B (e.g., EC 3.4.23.2) and Progastricin.

As mentioned, large peptides generated by the action of pepsin, are broken down further in the intestine into smaller peptides and free amino acids by proteases of the enteropeptidase pathway (see FIG. 1).

Thus, according to a second embodiment of the present invention, the method is effected by down-regulating at least one component of the enteropeptidase pathway (i.e., activators of enteropeptidase, enteropeptidase itself and downstream effectors of enteropeptidase, e.g., see FIG. 1), which governs intestinal protein degradation and pancreatic lipase activation, thereby allowing inhibition of energy absorption deriving from proteins and from triglycerides.

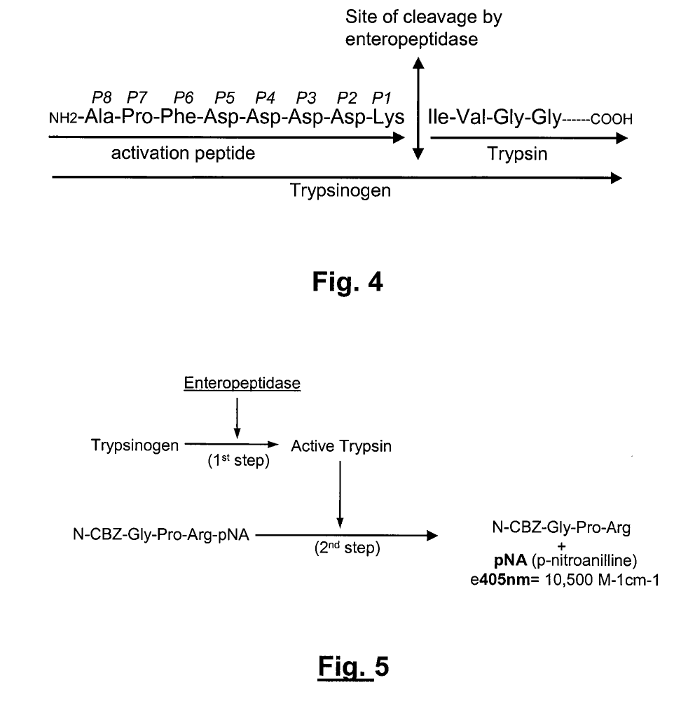

As used herein “enteropeptidase” refers to a heterodimeric serine protease that activates trypsins and downstream proteases (e.g., EC 3.4.21.9). The serine protease enteropeptidase (EP, also termed enterokinase) is present in the duodenal and jejunal mucosa and is involved in the second phase of digestion of dietary proteins. Specifically, EP catalyzes the conversion, in the duodenal lumen, of trypsinogen into active trypsin via the cleavage of the acidic propeptide from trypsinogen. The activation of trypsin initiates a cascade of proteolytic reactions leading to the activation of many pancreatic zymogens. [See FIG. 1 and Antonowicz, Ciba Found. Symp., 70: 169-187 (1979); Kitamoto et al., Proc. Natl. Acad. Sci. USA, 91(16): 7588-7592 (1994)]. EP is highly specific for the substrate sequence (Asp)₄-Lys-Ile present in the trypsinogen molecule, where it acts to mediate cleavage of the Lys-Ile bond (FIG. 4).

Enteropeptidase is a disulfide-linked heterodimer composed of a heavy chain of 82-140 kDa, and a light chain of 35-62 kDa [Mann (1994) Proc. Soc. Exp. Biol. Med. 206:114-8]. Mammalian EPs contain 30-50% carbohydrates, which may contribute to the apparent differences in its peptide weight. The heavy chain is postulated to mediate association with the intestinal brush border membrane [Fonseca (1983) J. Biol. Chem. 258:14516-14520], while the light chain contains the catalytic site localized in the intestine lumen. Nucleotide and protein Accession numbers (according to NCBI) of enteropeptidase from different organisms are given in Table 1.

TABLE 1

| | | | Protein size |
|---|---|---|---|
| Organism | Nucleotide | Protein | (in amino acid) |
| H. sapiens | NM_002772 | NP_002763 | 1019 |
| P. troglodytes | XM_514836 | XP_514836 | 1089 |
| C. familiaris | XM_544824 | XP_544824 | 1034 |
| M. musculus | NM_008941 | NP_032967 | 1069 |
| R. norvegicus | XM_213668 | XP_213668 | 1042 |
| B. taurus | NM_174439 | NP_776864 | 1035 |
| S. scrofa | NM_001001259 | NP_001001259 | 1034 |
| G. gallus | XM_425539 | XP_425539 | 1044 |

As used herein a “downstream effector” refers to a target molecule in a pathway. The downstream effectors of enteropeptidase include the serine proteases trypsins (e.g., EC 3.4.21.4), chymotrypsin (e.g., EC 3.4, 21.1), elastases (e.g., EC 3.4.21.36), and the metalloproteases carboxypeptidase A, carboxypeptidase B and pancreatic lipase and zymogens thereof, as well as, enzymes participating in the hydrolysis of small peptides such as aminopeptidases (e.g., EC 3.4.11.2), dipeptidases (e.g., EC 3.4.13.18) and tripeptidases (EC 3.4.11.4). Nucleotide and protein Accession numbers of tryspin from different organisms are given in Table 2.

TABLE 2

| | | | Protein size |
|---|---|---|---|
| Organism | Nucleotide | Protein | (in amino acid) |
| H. sapiens | NM_002769 | NP_002760 | 247 |
| P. troglodytes | XM_519441 | XP_519441 | 247 |
| C. familiaris | XM_532744 | XP_532744 | 246 |
| M. musculus | NM_053243 | NP_444473 | 246 |
| R. norvegicus | NM_012729 | NP_036861 | 246 |
| B. taurus | NM_174690 | NP_777115 | 247 |
| G. gallus | AAN75630 | AF110982 | 248 |

An example of an activator of enteropeptidase is the serine protease, duodenase [Zamolodchikova et al., 1995 Eur J Biochem 227, 866-872]. Since duodenase and enteropeptidase control this important protein digestive pathway in addition to the pancreatic lipase activity, agents which are directed at either or both of these targets are currently preferred according to this aspect of the present invention, to avoid redundancy.

Agents capable of down-regulating activity or expression of proteins or mRNA transcripts encoding thereof are well known in the art.

Since many of the protein targets of the present invention are localized in the lumen of the small intestine, which is featured by high protease activity, agents of the present invention (e.g., protein agents) are preferably modified to increase bioavailability thereof. Thus, agents of the present invention may be chemically modified to improve their stability. Agents of the present invention may be administered using bioadhesive delivery systems capable of enhancing contact of the drug with the mucous membrane lining the gastro-intestinal tract. Furthermore, the use of carrier systems such as micro-spheres and nanoparticles that can improve the bioavailability of the agents may be preferred [see Pappas (2004) Expert Opin. Biol. Ther. 4:881-7; Cefalu (2004) Drugs 64:1149-61; and Gowthamarajan and Kulkami (2003) Resonance 38-46].

For example, agents of the present invention, including protease inhibitors, oligonucleotides, antibodies, antibody fragments and non functional derivatives of the components of the pathways discussed herein are preferably combined with a mucoadhesive agent in order to improve drug delivery. Various mucoadhesive agents, e.g., mucoadhesive polymers are known which are believed to bind to the mucus layers coating the stomach and other regions of the gastrointestinal tract. Examples of mucoadhesive polymers as discussed herein include, but are not limited to chitosan, polyacrylic acid, hydroxypropyl methylcellulose and hyaluronic acid. Most preferably, the mucoadhesive polymer is chitosan [Guggi et al., (2003) J of Controlled Release 92:125-135].

In one preferred embodiment, the agent is a protease inhibitor, which is designed to specifically inhibit the activity or the expression of a particular protease participating in protein digestion and/or absorption (see above). For example, when the protease target is of the enteropeptidase pathway, a serine protease inhibitor is preferably used. Particularly interesting are protease inhibitor having a cumulative effect on both enteropeptidase and trypsin i.e., agents that are able to inhibit both enteropeptidase and trypsin activities. Also concerned are protease inhibitors having an inhibitory effect on enteropeptidase or trypsin only. When the down-regulated protease is pepsin, an aspartic protease inhibitor is required. A synthetic protease inhibitor, such as camostat, may also be used.

Aspartic protease inhibitors which can be utilized by the present invention are well known in the art. Examples include, but are not limited to, naturally occurring or synthetic, low or high molecular weight inhibitors including peptide or non-peptide based inhibitors. As used herein, a low molecular weight inhibitor is one which is typically below one kilodalton.

Aspartic protease inhibitors which can be utilized by the present invention to inhibit pepsin include, but are not limited to, the high molecular weight synthetic peptidomimetic protease inhibitors. The mechanism of action of these peptide-based inhibitors involves the formation of a transition-state analogue. Examples of peptidomimetic protease inhibitors of pepsin include retroviral protease inhibitors which are typically utilized in the treatment of human immunodeficiency virus (HIV) and hepatitis C virus (HCV).

Examples of retroviral protease inhibitors which can be utilized by the present invention to inhibit pepsin include, but are not limited to, CGP 53437, Amprenavir, Atazanavir, Indinavir, Lopinavir, Fosamprenavir, Nelfinavir, Ritonavir and Saquinavir.

CGP 53437 which demonstrates the highest affinity for pepsin is presently preferred (Kᵢ=8 nM) [Alteri (1993) Antimicrob. Agents. Chemother. 37:2087-92]. It should be noted that retroviral protease inhibitors which demonstrate low bioavailability and remain in the gastrointestinal lumen are also preferred since use thereof should reduce potential side effects due to the inhibition of other aspartic proteases such as Cathepsin D and β secretase.

Typically, low molecular weight aspartic protease inhibitors irreversibly modify an amino acid residue on the protease active site. One example of a low molecular weight aspartic protease inhibitors which can be utilized by the present invention is pepstatin A. This protease inhibitor is a pentapeptide with a molecular weight of 686 Daltons. It is naturally occurring, secreted by Streptomyces bacteria. It is a potent inhibitor of various aspartic proteases including the cathepsin D, the renin, the pepsins, bacterial aspartic proteases and the HIV protease. The prolonged retention in the stomach at the required site of action, by linking pepstatin to a mucoadhesive polymer, is especially important since it is a small non-specific molecule. Immobilization has the benefit of both slowing clearance from the body and minimizing systemic side effects of the protease inhibitors.

Naturally occurring protease inhibitors have been isolated in a variety of organisms from bacteria to animals and plants. Generally, these behave as tight-binding reversible or pseudo-irreversible inhibitors of proteases preventing substrate access to the active site through steric hindrance. Their sizes typically range from 50 residues (e.g. BPTI: Bovine Pancreatic Trypsin Inhibitor) to 400 residues (e.g. alpha-1PI: alpha-1 Protease Inhibitor) and they are strictly class-specific.

Examples of natural aspartyl protease inhibitors other than pepstatin include, but are not limited to, extracts from solanum tuberosum (potato), Cucurbita maxima (squash) and Anchusa strigosa (Prickly Alkanet) [Strukelj (1990) Nuc. Acid. Res. 18:4605; Farley (2002) J. Mol. Recognit. 15:135-44; Abuereisch (1998) Phytochemistry 48:217-21]. Other potent natural aspartyl protease inhibitors are those originally isolated from the round worms, Ascaris suum and Ascaris lumbricoides. These natural protease inhibitors include, but are not restricted, Pespin inhibitor III (PI-3) that inactivate pepsins and cathepsin E. These inhibitors are believed to protect the worm from gastric aspartic proteases in the stomach of their host [Abu-Ereish (1974) J. Biol. Chem. 249:1566-71; Kageyama (1998) Eur. J. Bioch. 253:804-9].

Serine protease inhibitors can be used to inhibit the activity of components of the enteropeptidase pathway, as well. These include low or high molecular weight inhibitor groups.

Either synthetic or of bacterial and fungal origin, small serine protease inhibitors irreversibly modify an amino acid residue on the protease active site. Examples of low molecular weight serine protease inhibitors include, but are not limited to, E-64 [Matsushima (1999) Biochem. 125:947-51], antipain, elastatinal, leupeptin, PMSF and its derivative APMSF, benzamidine and its derivative p-aminobenzamidine, chymostatin, TLCK, TPCK, DFP and 3,4-dichloroisocoumarin, all of which are commercially available.

An example of a high molecular weight serine protease inhibitor is the non-peptide based orally active inhibitor of elastase is−(9-(2-piperidinoethoxy)-4-oxo-4H-pyrido 1,2-a pyrimidin-2-yloxymet-hyl)-4-(1-methylethyl)-6-methoxy-1,2-benzisothiazol-3(2H)-one-1,1-dioxide (SSR69071) [Kapui (2003) Pharmacol Exp Ther 305:451-9].

An example of a peptide based inhibitor of enteropeptidase is Val-(Asp)4-Lys-chloromethyl ketone artificial inhibitor [Antonowicz (1980) Clin. Chim. Acta. 101(1):69-76; Lu (1999) 17:292:361-73].

Natural inhibitors of the enteropeptidase pathway can also be employed by the present invention. Natural serine protease inhibitors are presently the most widely studied of all naturally occurring protease inhibitors. Examples include, α1-protease inhibitor that can be used to inhibit duodenase [Gladysheva (2001) Biochemistry 66(6): 682-7]. Other examples of natural duodenase inhibitors include the inhibitors of the Bowman-Birk family [Gladysheva (2002) Protein Pept. Lett. 9(2): 139-44]. Natural inhibitors of trypsin include the soybean trypsin inhibitor, and extracts of black and white garden beans [Rascon (1985) Comp. Biochem. Physiol. B. 82:375-8]. Natural inhibitors of enteropeptidase include lectins [Rouanet (1983) Experimentia 39:1356-8], kidney bean inhibitors [e.g., EPI purified from red kidney bean, Jacob (1983) Biochem J. 209:91-7; Tajiri (1986) J. Nutr. 116:873-80] and DI which is isolated from bovine duodenum [Mikhailova (1998) Vopr. Med. Khim. 44:338-46].

Another example of an agent capable of downregulating a protein component participating in protein digestion and/or absorption is an antibody or antibody fragment capable of specifically binding the protease, preferably to its active site, thereby preventing its function. For example, amino acids 801-1035 of bovine enteropeptidase, have been determined as its active site [Kitamoto (1994), Proc. Natl. Acad. Sci. USA 91:7588-7592].

The 3 D structure of pepsin renders this protease a good target for antibody manipulation. The antibody can be targeted against its active cleft between its two domains. A flap located over the active site cleft which allows substrate access is another target for antibody recognition [Zlabinger G J et al., Matrix. 1989 (2):135-9].

Preferably, the antibody specifically binds to at least one epitope of the protein. As used herein, the term “epitope” refers to any antigenic determinant on an antigen to which the paratope of an antibody binds. Epitopes of enteropeptidase catalytic domain preferably include His841, Asp892 and Ser987 [Kitamoto 1994, Proc. Natl. Acad. Sci. USA 91:7588-7592].

Epitopic determinants usually consist of chemically active surface groups of molecules such as amino acids or carbohydrate side chains and usually have specific three-dimensional structural characteristics, as well as specific charge characteristics.

The term “antibody” as used in this invention includes intact molecules as well as functional fragments thereof, such as Fab, F(ab′)2, and Fv that are capable of binding to the antigen presented by the macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)2 is a dimer of two Fab′ fragments held together by two disulfide bridges; (4) Fv, defined as a genetically engineered fragment containing the variable region of the light chain and the variable region of the heavy chain expressed as two chains; and (5) Single Chain Antibody (“SCA”), a genetically engineered molecule containing the variable region of the light chain and the variable region of the heavy chain, linked by a suitable peptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., [Proc. Natl. Acad. Sci. USA 69:2659-62 (1972)]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single peptide chain with a linker peptide bridging the two V domains. Methods for producing sFvs are described in the litterature [Whitlow and Filpula [(1991), Methods 2: 97-105]; Bird et al., [(1988) Science 242:423-426]; Pack et al., [(1993), BioTechnology 11:1271-77]; and U.S. Pat. No. 4,946,778, which is hereby incorporated by reference.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [(1991) Human Antibodies and Hybridomas, 2:172-189 and U.S. Pat. No. 6,580,016].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source that is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1992); Marks et al., J. Mol. Biol., 222:581 (1991)]. The techniques of Cole et al., and Boemer et al., are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,806; 5,545,807; 5,569,825; 5,625,126; 5,633,425; 5,661,016, and in the following scientific publications: Marks et al., BioTechnology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Alternatively, the agent of this aspect of the present invention may be an oligonucleotide directed against an endogenous nucleic acid sequence expressing the at least one component participating in protein digestion and/or absorption.

In another embodiment, this oligonucleotide (DNA or RNA) is 15 to 30 base pair (bp), preferably 18 to 25 bp long and most preferably 21 bp in length. A oligonucleotide according to the invention is a nucleic acid sequence complementary to the sequences of enteropeptidase or trypsin, and particularly the sequence indicated in Tables 1 and 2. The term “complementary” as defined herein means an oligonucleotide that hybridizes with the sequence to target under its entire length, under stringent conditions (for example, an hybridization carried out between 35 to 65° C. using a salt solution which is about 0.9 M). The hybridization may be perfect (100% matching) or imperfect with a mismatch in 1, 2 or 3 nucleotides. An oligonucleotide with some mismatches is considered to be appropriate for the invention if it can direct the degradation of the mRNA, which it is hybridized to.

In a first embodiment, the oligonucleotide is complementary to SEQ ID NO:3 (nucleic acid sequence encoding the human enteropeptidase; SEQ ID NO:4) or a homologue thereof (Table 1). In a second embodiment, the oligonucleotide is complementary to SEQ ID NO:1 (nucleic acid sequence encoding the human trypsin; SEQ ID NO:2) or a homologue thereof (Table 2).

A small interfering RNA (siRNA) molecule is an example of an oligonucleotide agent capable of downregulating a component participating in protein digestion and/or absorption. RNA interference is a two-step process. During the first step, which is termed the initiation step, input dsRNA is digested into 21-23 nucleotides (nt) small interfering RNAs (siRNA), probably by the action of Dicer, a member of the RNase III family of dsRNA-specific ribonucleases, which cleaves dsRNA (introduced directly or via an expressing vector, cassette or virus) in an ATP-dependent manner. Successive cleavage events degrade the RNA to 19-21 bp duplexes (siRNA), each strand with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al., (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs, which would generate more siRNAs, or by replication of the siRNAs formed. Alternatively or additionally, amplification could be effected by multiple turnover events of the RISC [Hammond et al., Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the mRNA sequence target is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and significantly reduced protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites that exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. A G/C content comprised between 30 to 50% is preferred. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another oligonucleotide agent capable of downregulating a component participating in protein digestion and/or absorption is a DNAzyme molecule capable of specifically cleaving an mRNA transcript or a DNA sequence of the target. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 94:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-ab1 oncogenes were successful in inhibiting the oncogenes expression in leukemia cells, and lessening relapse rates in autologous bone marrow transplant in cases of Chronic Myelogenous Leukemia (CML) and Acute Lymphocytic Leukemia (ALL).

Downregulation of a component participating in protein digestion and/or absorption can also be effected by using an antisense polynucleotide capable of specifically hybridizing with an mRNA transcript encoding the component participating in protein digestion and/or absorption (e.g., a 21 antisense oligonucleotide directed at the specific enteropeptidase site R₉₆RRK₉₉ which is located in the light (catalytic) chain of enteropeptidase).

Design of antisense molecules, which can be used to efficiently down-regulate a component participating in protein digestion and/or absorption, must be effected while considering two aspects important to the antisense approach. The first aspect is delivery of the oligonucleotide into the cytoplasm of the appropriate cells, while the second aspect is design of an oligonucleotide that specifically binds the designated mRNA within cells in a way that inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al., Blood 91: 852-62 (1998); Rajur et al., Bioconjug Chem 8: 935-40 (1997); Lavigne et al., Biochem Biophys Res Commun 237: 566-71 (1997) and Aoki et al., (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al., Biotechnol Bioeng 65: 1-9 (1999)].

Such algorithms have been successfully used to implement an antisense approach in cells. For example, the algorithm developed by Walton et al., enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved to be effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Homlund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a component participating in protein digestion and/or absorption is a ribozyme molecule capable of specifically cleaving an mRNA transcript encoding a component participating in protein digestion and/or absorption. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—http://www.rpi.com/index.html).

An additional method of regulating the expression of a component participating in protein digestion and/or absorption genes in cells is via triplex forming oligonucleotides (TFOs). In the last decade, studies have shown that TFOs can be designed which can recognize and bind to polypurine/polypirimidine regions in double-stranded helical DNA in a sequence-specific manner. These recognition rules are outlined by Maher III, L. J., et al., Science (1989) 245:725-730; Moser, H. E., et al., Science (1987)238:645-630; Beal, P. A., et al., Science (1991) 251:1360-1363; Cooney, M., et al., Science (1988)241:456-459; and Hogan, M. E., et al., EP Publication 375408. Modification of the oligonucleotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer (2003) J Clin Invest; 112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch (2002), BMC Biochem, Sept. 12, Epub). The same authors have demonstrated that TFOs designed according to the A-AT and G-GC rule do not form non-specific triplexes, indicating that the triplex formation is indeed sequence specific.

Thus for any given sequence in the regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFG1 and endogenous HPRT genes in mammalian cells (Vasquez et al., Nucl Acids Res. (1999) 27:1176-81, and Puri, et al., J Biol Chem, (2001) 276:28991-98), and the sequence- and target-specific downregulation of expression of the Ets2 transcription factor, important in prostate cancer etiology (Carbone, et al., Nucl Acid Res. (2003) 31:833-43), and the pro-inflammatory ICAM-1 gene (Besch et al., J Biol Chem, (2002) 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res (2000); 28:2369-74).

Additionally, TFOs designed according to the abovementioned principles can induce directed mutagenesis capable of effecting DNA repair, thus providing both downregulation and upregulation of expression of endogenous genes [Seidman and Glazer, J Clin Invest (2003) 112:487-94]. Detailed description of the design, synthesis and administration of effective TFOs can be found in U.S. Patent Application Nos. 2003 017068 and 2003 0096980 to Froehler et al., and 2002 0128218 and 2002 0123476 to Emanuele et al., and U.S. Pat. No. 5,721,138 to Lawn.

Additional description of oligonucleotide agents is further provided hereinbelow. It will be appreciated that therapeutic oligonucleotides may further include base and/or backbone modifications, which may increase bioavailability, therapeutic efficacy and reduce cytotoxicity. Such modifications are described in Younes (2002) Current Pharmaceutical Design 8:1451-1466.

For example, the oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described herein below.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidates and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e. the backbone, of the nucleotide units are replaced with novel groups. The base units are maintained for complementation with the appropriate polynucleotide target. An example for such an oligonucleotide mimetic includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al., (1993) Antisense Research and Applications, CRC Press, Boca Raton 276-278] and are presently preferred base substitutions, even more particularly when combined with 2′-O-methoxyethyl sugar modifications.

Examples of oligonucleotide agents which have been used to down-regulate expression of duodenal proteins are described in Ratineau (2004) J. Biol. Chem. 279:24477-84; Khomenko (2003) Biochem. Biophys. Res. Commun. 309:910-6; Morel (1997) Br. J. Pharmacol. 121:451-8.

Alternatively, an agent capable of down-regulating the activity of a component participating in protein digestion and/or absorption can be a non-functional derivative thereof (i.e. dominant negative). Enteropeptidase forms, which include mutations that render the protein inactive, are known in the art [Holzinger (2002) Am. J. Hum. Genet. 70(1):20-5]. These mutations include, for example, the nonsense mutations S712X, R857X and Q261X, as well as the frameshift mutation FsQ902. At least one of these mutations can be introduced to the subject using the well known “gene knock-in strategy” which will result in the formation of a non-functional protein [see e.g., Matsuda et al., Methods Mol. Biol. 2004; 259:379-90]. Alternatively, a non-functional derivative of enteropeptidase can be provided to the subject. Such derivatives may have altered membrane localization, or substrate specificity [Kitamoto (1994) Proc. Natl. Acad. Sci. USA 91:7588-7592].

The amino acid sequence of pepsin together with its 3-D structure makes pepsin a relatively easy target for point mutations and gene knock-in strategy. The enzyme is made up of two domains each of which contributes one aspartic acid residue to the catalytic site. These residues are essential in coordinating a water molecule for nucleophilic attack on the scissile peptide bond. Thus a point mutation in either of these aspartic acid residues would render the protease inactive and could be introduced to the subject using the gene knock-in approach as mentioned herein. An example of a pepsin mutation known in the art includes T77V [Okoniewska et al., Protein Engineering, 1999; 12: 55-61].

Peptides of these non-functional derivatives can be synthesized using solid phase peptide synthesis procedures that are well known in the art and further described by John Morrow Stewart and Janis Dillaha Young, [Solid Phase Peptide Syntheses (2nd Ed., Pierce Chemical Company, 1984)]. Synthetic peptides can be purified by preparative high performance liquid chromatography [Creighton T. (1983) Proteins, structures and molecular principles. WH Freeman and Co. N.Y.] and the composition of which can be confirmed by amino acid sequencing.

In cases where large amounts of the peptide are desired, they can be generated using recombinant techniques such as described by Bitter et al., (1987) Methods in Enzymol. 153:516-544, Studier et al., (1990) Methods in Enzymol. 185:60-89, Brisson et al., (1984) Nature 310:511-514, Takamatsu et al., (1987) EMBO J. 6:307-311, Coruzzi et al., (1984) EMBO J. 3:1671-1680 and Brogli et al., (1984) Science 224:838-843, Gurley et al., (1986) Mol. Cell. Biol. 6:559-565 and Weissbach & Weissbach, 1988, Methods for Plant Molecular Biology, Academic Press, NY, Section VIII, pp 421-463.

Alternatively, these peptides can be manufactured within the target cell by administering a nuclear acid construct of the peptide. It will be appreciated that the nucleic acid construct can be administered to the individual employing any suitable mode of administration, described hereinbelow (i.e. in vivo gene therapy). Alternatively, the nucleic acid construct can be introduced into a suitable cell using an appropriate gene delivery vehicle/method (transfection, transduction, etc.) and an appropriate expression system. The modified cells are subsequently expanded in culture and returned to the individual (i.e. ex vivo gene therapy). Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and transcription of the transgene is directed from the CMV promoter. Vectors derived from Mo-MuLV are also included such as pBabe, where the transgene will be transcribed from the 5′LTR promoter.

Currently preferred in vivo nucleic acid transfer techniques include infection with viral or transfection with a non-viral constructs. The former includes, but is not limited to the adenovirus, lentivirus, Herpes simplex I virus and adeno-associated virus (AAV) whilst the latter includes, but is not limited to lipid-based systems. Useful lipids for lipid-mediated transfer of the gene are, for example, DOTMA, DOPE, and DC-Chol [Tonkinson et al., Cancer Investigation, 14(1): 54-65 (1996)]. Recently, it has been shown that Chitosan can be used to deliver nucleic acids to the intestine cells (Chen J. (2004) World J Gastroenterol 10(1): 112-116). The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-transcriptional modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably, the signal sequence for this purpose is a mammalian signal sequence or the signal sequence of the peptide variants of the present invention. Optionally, the construct may also include a signal that directs polyadenylation, as well as one or more restriction site and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

As mentioned hereinabove, agents of the present invention can be used for reducing body fat content and as such can be used for treating conditions or disorders associated directly or indirectly with abnormal fat metabolism. Examples include, but are not limited to, overweight, obesity (i.e. at least 20% over the average weight for the person's age, sex and height), type II diabetes, hyperglycemia, hyperinsulinemia, elevated blood levels of fatty acids or glycerol, syndrome X, diabetic complications, dysmetabolic syndrome and related diseases, sexual dysfunction, hypercholesterolemia, atherosclerosis, hypertension, pancreatitis, hypertriglyceridemia, hyperlipidemia, Alzheimer's disease, osteopenia, stroke, dementia, coronary heart diseases, peripheral vascular diseases, peripheral arterial diseases, vascular syndromes, reducing myocardial revascularization procedures, microvascular diseases (e.g., neuropathy, nephropathy and retinopathy), nephritic syndrome, cholesterol-related disorders (e.g., LDL-pattern B and LDL-pattern L), cerebrovascular diseases, malignant lesions (e.g., ductal carcinoma in situ), premalignant lesions, gastrointestinal malignancies (e.g., liposarcoma, epithelial tumors, irritable bowel syndrome, Crohn's disease, gastric ulceritis, gallstones), drug-induced lipodystrophy, inflammatory disorders and climacteric. Agents of the present invention may also be used to treat non-diabetis obesity or non-pancreatitis patients.

It will be appreciated that the agents of the present invention may also be used to modulate body fat content. Thus, for example, agents of the present invention can be used to reduce percent body fat as is often desired by athletes.

As used herein the term “treating” refers to preventing, curing, reversing, attenuating, alleviating, minimizing, suppressing or halting the deleterious effects of a condition or disorder associated with abnormal fat metabolism symptoms and/or disease state.

The present invention also envisages treating subjects suffering from diseases, in which low-protein diet is typically recommended (in order to reduce symptoms of the disease and make the disease more manageable) with agents of the present invention. Examples of such diseases include, but are not limited to, renal diseases (e.g., chronic renal failure) Parkinson's disease [Riley (1988) Neurology 38:1026-31], Phenylketonuria (PKU), osteoporosis, alkaptonuria (AKU), liver diseases (www.gicare.com/pated/edtgs10.htm), urea cycle disorders and gout (www.cbsnews.com/stories/2004/03/11/health/main605445.shtml).

As used herein in the specification and claims section that follows, the phrase “therapeutically effective amount” refers to an amount which improves at least one of the following criteria: body mass index; % body fat; total body potassium, bioelectrical impedence or under water weighing. As used herein, the body mass index is the ratio between weight (in kilograms) and height squared (in meters square). Total body potassium, which is largely intracellular, is ascertained using a method to detect the natural decay of potassium 40 to potassium 39. This can be used to calculate lean body mass which when subtracted from total body weight will yield body fat mass. The total body potassium method is not widely available for clinical use because it necessitates a spectrometry measurement.

The criteria of bioelectric impedence as used herein is measured using a portable device with paste electrodes which are attached to the right hand and foot. With the patient supine, the total body electrical impedance or resistance is measured. Since water conducts electricity while fat is an insulator, the machine measures body water and calculates body fat. Another method for detecting fat body mass is “Underwater weighing”. This method relies on the fact that fat floats in water. Therefore, by comparing body weight on land and underwater, percent body fat can be calculated. Since air also floats, a correction must be made for lung volume, and subjects are encouraged to exhale as they put their heads underwater. This method is especially useful calculating fat body mass in athletes.

The “therapeutically effective amount” will, of course, be dependent on, but not limited to the subject being treated, the severity of the anticipated affliction, the manner of administration, as discussed herein and the judgment of the prescribing physician. [See e.g. Fingl, et al., (1975) “The Pharmacological Basis of Therapeutics”, Ch. 1 p. 1].

Determination of a therapeutically effective amount is well within the capability of those skilled in the art. Daily conventional dosages for protease inhibitors may be between 100 to 2000 mg, preferably 500 to 1500 mg, 800 to 1200 mg and most preferably between 800 and 1200 mg, in several timers daily.

For any preparation used in the methods of the invention, the therapeutically effective amount or dose can be estimated initially from in vitro assays. For example, a dose can be formulated in animal models (e.g. obese models such as disclosed by Bayli's J Pharmacol Exp Ther. 2003; and models for atherosclerosis such as described by Brousseau J Lipid Res. (1999) 40(3):365-75 and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human.

Depending on the severity and responsiveness of the condition to be treated, dosing can be effected over a short period of time (i.e. several days to several weeks) or until cure is effected or diminution of the disease state is achieved.

Agents of the present invention can be provided to the subject per se, or as part of a pharmaceutical composition where they are mixed with a pharmaceutically acceptable carrier.

As used herein a “pharmaceutical composition” refers to a preparation of one or more of the active ingredients described herein (i.e. agents) with other chemical components such as physiologically suitable carriers and excipients. The purpose of a pharmaceutical composition is to facilitate administration of a compound to an organism.

Herein the term “active ingredient” refers to the agent preparation, which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al., (1979).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections. The preferred route of administration is presently oral.

Carrier systems such as micro-spheres and nanoparticles that can improve the bioavailability of the agents may be preferably used in conjunction with the present invention [see Pappas (2004) Expert Opin. Biol. Ther. 4:881-7; Cefalu (2004) Drugs 64:1149-61; and Gowthamarajan and Kulkarni (2003) Resonance 38-46]. Additionally, microemulsion formulations offer improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity [Constantinides et al., Pharmaceutical Research, 1994, 11:1385; Ho et al., J. Pharm. Sci., 1996, 85:138-143].

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize, wheat, rice, or potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents that increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Compositions including the preparation of the present invention formulated in a compatible pharmaceutical carrier may also be prepared, placed in an appropriate container, and labeled for treatment of an indicated condition.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

As mentioned hereinabove, agents of the present invention may also be used for reducing body fat content in animals such as domestic animals. In this case agents of the present invention may be administered, dispersed in, or mixed with, animal feedstuff, drinking water and other liquids normally consumed by the animals, or in compositions containing the agents of the present invention dispersed in or mixed with any other suitable inert physiologically acceptable carrier or diluent which is preferably orally administrable (as defined hereinabove). Such compositions may be administered in the form of powders, pellets, solutions, suspensions and emulsions, to the animals to supply the desired dosage of the agents of the present invention or used as concentrates or supplements to be diluted with additional carrier, feed-stuff, drinking water or other liquids normally consumed by the animals, before administration. Suitable inert physiologically acceptable carriers or diluents include wheat flour or meal, maize gluten, lactose, glucose, sucrose, talc, kaolin, calcium phosphate, potassium sulphate and diatomaceous earths such as keiselguhr. Concentrates or supplements intended for incorporation into drinking water or other liquids normally consumed by the animals to give solutions, emulsions or stable suspensions, may also include the active agent in association with a surface-active wetting, dispersing or emulsifying agent such as Teepol, polyoxyethylene (20) sorbitan mono-oleate or the condensation product of β-naphthalenesulphonic acid with formaldehyde, with or without a physiologically innocuous, preferably water-soluble, carrier or diluent, for example, sucrose, glucose or an inorganic salt such as potassium sulphate, or concentrates or supplements in the form of stable dispersions or solutions obtained by mixing the aforesaid concentrates or supplements with water or some other suitable physiologically innocuous inert liquid carrier or diluent, or mixtures thereof (see U.S. Pat. No. 4,005,217).

Each of the agents described hereinabove is administered to the treated subject for a time period sufficient to prevent degradation of essential proteins which may be life threatening (see Guyton and Hall “The Textbook of Medical Physiology” 10^{th} Ed. Harcourt International Edition).

It will be appreciated that the agents of the present invention may be administered in combination with other drugs to achieve enhanced effects (e.g., see Background section and WO 2004/037159 to Harosh).

It will be further appreciated that the agents of the present invention may also be provided as food additives.

The phrase “food additive” [defined by the FDA in 21 C.F.R. 170.3(e)(1)] includes any liquid or solid material intended to be added to a food product. This material can, for example, include an agent having a distinct taste and/or flavor or a physiological effect (e.g., vitamins).

The food additive composition of the present invention can be added to a variety of food products.

As used herein, the phrase “food product” describes a material consisting essentially of protein, carbohydrate and/or fat, which is used in the body of an organism to sustain growth, repair and vital processes and to furnish energy. Food products may also contain supplementary substances such as minerals, vitamins and condiments. See Merriani-Webster's Collegiate Dictionary, 10th Edition, 1993. The phrase “food product” as used herein further includes a beverage adapted for human or animal consumption.

A food product containing the food additive of the present invention can also include additional additives such as, for example, antioxidants, sweeteners, flavorings, colors, preservatives, nutritive additives such as vitamins and minerals, amino acids (i.e. essential amino acids), emulsifiers, pH control agents such as acidulants, hydrocolloids, antifoams and release agents, flour improving or strengthening agents, raising or leavening agents, gases and chelating agents, the utility and effects of which are well-known in the art.

The present invention also concerns a composition comprising an agent capable of down-regulating activity and/or expression of at least one component participating in protein digestion and/or absorption as defined above, for use in the reduction of percent body fat or for treating conditions or disorders associated directly or indirectly with abnormal fat metabolism.

Moreover, the use of an agent capable of down-regulating activity and/or expression of at least one component participating in protein digestion and/or absorption as defined above, in the manufacture of a composition or a drug for the treatment of conditions or disorders associated directly or indirectly with abnormal fat metabolism also is part of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### EXAMPLES

### Example 1

In vitro Testing: the Trypsinogen Activation Assay

Material: The following component, used in the present trypsinogen activation assay may be purchased as follows:

TABLE 3

| Component | Purchaser; catalogue number |
|---|---|
| Recombinant human enteropeptidase | R&D Systems; 1585-SE. |
| N-CBZ-Gly-Pro-Arg-pnitroanilide | SIGMA; C2276 |
| Trypsinogen | SIGMA; T-1143 |
| AC-Leu-Val-Lys-Aldhehyde⁽²⁾ | Bachem; N-1380 (4020266) |
| BOC-Ala-Glu-Val-Asp-Aldehyde⁽¹⁾ | Bachem; N-1755 (4029153) |
| H-D-Tyr-Pro-Arg-chloromethylketone | Bachem; N-1225 (40173722) |
| trifluroroacetate salt ⁽²⁾ | |
| Z-Asp-Glu-Val-Asp- | Bachem; N-1580 (4027524) |
| chloromethylketone ⁽²⁾ | |
| 1,5-Dansyl-Glu-Gly-Arg- | Calbiochem; 251700 |
| chloromethylketone | |
| dihydrochloride ⁽¹⁾ | |

⁽¹⁾ negative control;
⁽²⁾ candidate molecule

Method

The trypsinogen activation assay is shown in FIG. 5. In the first step, the enteropeptidase cleaves the trypsinogen in its active form, trypsin. Trypsin, in the second step, cleaves the N-CBZ-Gly-Pro-Arg-p-nitroanilide (pNA) into N-CBZ-Gly-Pro-Arg and p-nitroanilide (pNA). The amount of pNA can be measured at 405 nm, and reflects the amount of trypsin cleaved and thus the inhibitory activity of the molecules tested on the enteropeptidase.

In the first step, the following mix was prepared (50 μl final)
recombinant human enteropeptidase: 1.5 nM final
sodium citrate: 50 nM final
candidate molecule or control: 1 μl
trypsinogen: 2.5 μM final

The mix was incubated at room temperature during 10 minutes and the reaction was stopped with 5 μl of HCl 0.4 M.

In the second step, the previous mix was then incubated with a 50 μl mix comprising 1 mM of N-CBZ-Gly-Pro-Arg-pNA, Tris Hcl pH 8.4 20 mM final and NaCl-150 mM final, at room temperature for 10 minutes. The absorbance of the resulting mix was read at 405 nm.

Results are expressed as the percentage of inhibition, which is the absorbance at 405 nm of the reaction in the presence of different concentrations of inhibitor as compared to the value obtained in the absence of inhibitor.

Results

Control molecules (BOC-Ala-Glu-Val-Asp-Aldehyde and Z-Asp-Glu-Val-Asp-chloromethylketone) were tested at high concentration (10 and 50 μm respectively). As expected, no inhibition was observed, since these two molecules contain an aspartate residue at position P1 which is not expected to be recognised by enteropeptidase.

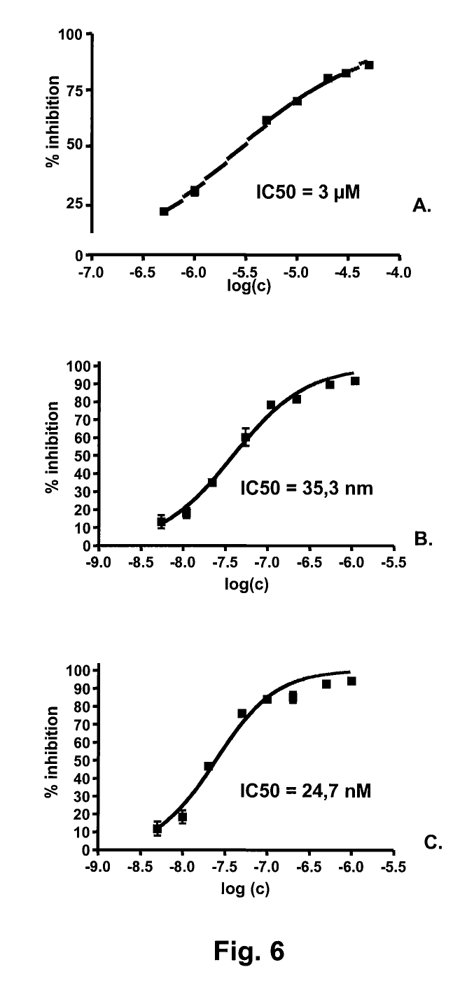

In contrast the three candidate molecules, tested for their suspected inhibition activity, show a 50% inhibition (as compared to values in absence of inhibitors) at very low concentrations. The IC50 measurement was performed using a Prism graphic application. Graphic representation and IC50 value for these candidate molecules are shown in FIG. 6A (AC-Leu-Val-Lys-Aldhehyde), FIG. 6B (H-D-Tyr-Pro-Arg-chloromethylketone trifluororoacetate salt) and FIG. 6C (1,5-Dansyl-Glu-Gly-Arg-chloromethylketone dihydrochloride).

The IC50 was about 3 μM for AC-Leu-Val-Lys-Aldhehyde, and about 35 and 24.7 nM for H-D-Tyr-Pro-Arg-chloromethylketone trifluororoacetate salt and 1,5-Dansyl-Glu-Gly-Arg-chloromethylketone dihydrochloride respectively.

Additional experiments have shown that H-D-Tyr-Pro-Arg-chloromethylketone trifluororoacetate salt and Z-Asp-Glu-Val-Asp-chloromethylketone molecules, when tested for enteropeptidase only, give a higher IC50 than the ones reported in FIG. 6 (data not shown).

Consequently, these observations show that candidate molecules able to compete with both trypsinogen and substrate of tryspin give a cumulative effect on inhibition of tryspin activity; first directly, by inhibiting the activity of tryspin, and also indirectly by inhibiting the activity of enteropeptidase.

Due to their low IC50 value, these molecules are excellent candidates for the enteropeptidase activity inhibition.

### Example 2

### In vivo Testing in Rats

To test the effects of molecules on the reduction of body fat, 30 male, genetically obese Zucker rats (Charles River Laboratories; strain: Crl: ZUC (Orl)-Lepr^{fa}) having an age of 16 weeks at the beginning of this study are utilized. Zucker rats have an autosomal recessive mutation that results in obesity. 30 Zucker rats are divided into 6 groups (5 rats in each group) of which:
1 group is used as a control and received water only;
1 group is given a mix of 5 particular antisense oligonucleotides (Table 5 below),
2 groups receive H-D-Tyr-Pro-Arg-chloromethylketone trifluororoacetate salt (two concentrations),
2 groups receive Z-Asp-Glu-Val-Asp-chloromethylketone (two concentrations).

The 5 groups (2 to 6) all receive the candidate molecules in the same vehicle (water). The treatment is administered orally (gavage) one time per day, 15 to 30 minutes before food intake during 28 consecutive days, under conditions indicated in Table 4.

TABLE 4

| | | Test | Concen- |
|---|---|---|---|
| | | item | tration |
| | | mg/kg | of test |
| Group | Test item | of rat | item |
| 1 | Vehicle (water) | / | / |
| 2 | Mix of oligonucleotides | 0.04⁽¹⁾ | 0.004 |
| 3 | H-D-Tyr-Pro-Arg-chloromethylketone | 1 | 0.1 |
| | trifluroroacetate salt | | |
| 4 | H-D-Tyr-Pro-Arg-chloromethylketone | 4 | 0.4 |
| | trifluroroacetate salt | | |
| 5 | Z-Asp-Glu-Val-Asp-chloromethylketone | 1 | 0.1 |
| 6 | Z-Asp-Glu-Val-Asp-chloromethylketone | 4 | 0.4 |

⁽¹⁾0.04 mg of each oligonucleotide/kg of rat

The period of acclimation lasts for the first five days wherein food is given ad libitum. After the initial acclimation period of five days, the next fourteen days, the rats are conditioned by restricting their food intake by a period of three hours per day. All rats are given a hyper-protein food of 25 to 30%. The rats are observed 1 time per day. Their weight is monitored every 3 days during the 14 day period.

Testing Specific Oligonucleotides in the Zucker Rat

The sequences of the oligonucleotides chosen for this study are sequences that are complementary to the enteropeptidase nucleic acid of the rat and should recognize, within the cell, the mRNA of rat enteropeptidase. This heterocomplex of RNA:oligonucleotide induces the activation of RNase H which degrades the RNA strand. The oligonucleotides have about 20 bases and are protected from degradation by nucleases due to the modification of type 2′-O methyl in position 5′ (m) of the three last nucleotides.

5 oligonucleotides are chosen from the sequence of enteropeptidase and the name is the first position of the sequence on the enteropeptidase. These sequences are set forth in Table 5 below:

TABLE 5

| | Name of | | |
|---|---|---|---|
| | oligo- | | |
| Number | nucleotide | Sequence (from 5 to 3′) | SEQ ID |
| 1 | ODN2053 | CCTGCCTGGGTGTCACTTCmCmAmC | 5 |
| 2 | ODN2154 | GCAGCAGACACCAGCCAGTCmAmAmU | 6 |
| 3 | ODN1160 | GTAGGATGCTCTGGTGGAmGmGmG | 7 |
| 4 | ODN2689 | CCCAGGGTGATTAGGCAGTGmCmAmC | 8 |
| 5 | ODN1527 | CCTGGCAGGGCTGTGGAATmCmCmC | 9 |

m: 2′O-methyl modification in position 5′

40 μg/kg of each of the above oligonucleotides are given to each of the Zucker rats orally for 28 days.

Testing Molecules

H-D-Tyr-Pro-Arg-chloromethylketone trifluororoacetate salt and Z-Asp-Glu-Val-Asp-chloromethylketone are ordered from Bachem, and are available under reference N-1225 (40173722) and N-1580 (4027524). H-D-Tyr-Pro-Arg-chloromethylketone trifluororoacetate salt has a molecular formula C₂₁H₃₁ClN₆O₄, a relative molecular weight of 466.97 and a degree of purity of 91%. Z-Asp-Glu-Val-Asp-chloromethylketone has a molecular formula of C₂₇H₃₅N4O₁₂Cl, a relative molecular weight of 643.10 and a degree of purity more than 95%.

H-D-Tyr-Pro-Arg-chloromethylketone trifluororoacetate salt is used in vivo as a candidate molecule, since it gives excellent IC50 in in vitro experiment.

Z-Asp-Glu-Val-Asp-chloromethylketone, shown to not inhibit the enteropeptisae and trypsin, is used as a side effect control. Indeed, the chloromethylketone group may irritate the esophagus, and thus reduce the amount of candidate molecule ingerate due to lesion. This molecule may therefore, in the absence of inhibition of enteropeptisae and trypsin, enable the distinction between a loss of weight due to the candidate molecule (in the case of the H-D-Tyr-Pro-Arg-chloromethylketone trifluororoacetate) and a loss of weight due to esophagus injury.

At day 14 and at the end of the 28 days each of the rats are bled. Total protein, total cholesterol, HDL, LDL, glucose and triglycerides are measured using kits from HORIBA.ABX (Montpelier, France), according to the manufacturer's instructions.

### Example 3

The same conditions as the one described in example 2 are used in this example. Male obese Zucker rats are administrated with one or combination (2, 3, 4 or 5) oligonucleotides disclosed in Table 5.

Each of the rats, that are administered oligonucleotide numbers 1 to 5 or combination thereof, experiences a decrease in the levels of total protein, total cholesterol, LDL, glucose and triglycerides as compared to the control group. An increase in HDL is observed in the rats that are administered oligonucleotide numbers 1 to 5 or combination thereof, as compared to the control group.

The final weight of the rats is also undertaken. The rats administered the oligonucleotides numbers 1 to 5 or combination thereof experience a reduction in weight loss as compared to that of the control.

### Example 4

### Treating Obesity

A group of obese men and women are used in this example. Obesity is determined by their body mass index (BMI) kg/m². A value of over 30 kg/m² or greater is considered to be obese. 10 females having an average age of 30 years and 10 males having an average age of 40 years are used in this example. All of the people have a body mass index of over 30 kg/m², and more particularly ranging from 30 to 35 kg/m², which is indicative of obesity.

The study group is advised to follow their normal routine concerning their eating habits and exercise patterns, which is recorded 1 month prior to this study and throughout this study.

5 females and 5 males are given a treatment of ritonavir at 600 mg taken twice a day. The other group of 5 females and 5 males is given a placebo twice a day. The treatment continued for 2 months. At the end of two months another body mass index is taken of the control group and the treated group. The body mass index of the treated group decreased by a factor of 3 kg/m² to 5 kg/m² at the end of the two month period; i.e., an average weight loss between 20 and 30 pounds, while the mass body index of the control group remained unchanged.

### Example 5

### Treating Obesity

The same study is done as in Example 4, however different protease inhibitors are used in this study such as amprenavir, atazanavir, indinavir, lopinavir, fosamprenavir, nelfinavir or saquinavir. A larger group study is undertaken using 20 females and 20 males having an average age of 38 years and having a body mass index ranging from 30 to 40 kg/m². 7 groups of 4 (2 females & 2 males) are given one of the following doses of protease inhibitors:

Group 1: Amprenavir: 1,200 mg twice a day

Group 2: Atazanavir: 400 mg once a day

Group 3: Indinavir: 800 mg every 8 hours

Group 4: Lopinavir: 399 mg twice a day

Group 5: Fosamprenavir: 1,400 mg two times a day

Group 6: Nelfinavir: 750 mg three times a day

Group 7: Saquinavir: 1,000 mg twice a day

The remaining group of 6 males and 6 females are given a placebo. The treatment continued for 2 months. At the end of two months another body mass index is taken of the control group and the treated group. The body mass index of the treated group decreases by a factor of 3 kg/m² to 5 kg/m² at the end of the two month period; i.e., an average weight loss between 20 and 30 pounds, while the mass body index of the control group remained unchanged.

### Example 6

### Treating Type II Diabetes

Type II diabetes is a disease in which the amount of insulin produced by the pancreas is inadequate to meet the body's needs and thus glucose, which is metabolized by insulin is not taken up normally from the blood into the body tissues. Therefore glucose in the blood rises. Type II diabetes is detected by a fasting glucose level of greater than 126 mg/dL measured on two occasions or one blood glucose level of greater than 200 mg/dL on one occasion or two random blood glucose levels of more than 200 mg/dL. Also a glucose tolerance test having a glucose level of more than 200 mg/dL 2 hours after drinking 75 grams of glucose also qualifies an individual as having Type II diabetes.

Two groups of 10 people are used in this study. The first given a treatment of ritonavir at 600 mg taken twice a day, while the other 10 people were given a placebo. The treatment continued for 2 months.

The study group is advised to follow their normal routine concerning their eating habits and exercise patterns, which is recorded 1 month prior to this study and throughout this study.

At the end of two months blood glucose levels and a glucose tolerance test are tested with all of the people in the study. The people given ritonavir have significantly reduced levels of blood glucose than those in the control group.

### Example 7

### Treating Type II Diabetes

The same study in Example 6 is conducted with a larger group of people having Type II diabetes. Each of the treated groups 1 to 7 is given as amprenavir, atazanavir, indinavir, lopinavir, fosamprenavir, nelfinavir or saquinavir in the same amounts as set forth in Example 3. The control group is given a placebo. At the end of two months another fasting (9-12 hours) lipid profile is taken. The people given amprenavir, atazanavir, indinavir, lopinavir, fosamprenavir, nelfinavir or saquinavir have significantly reduced levels of blood glucose than those in the control group.

### Example 8

### Treating Hyperlipidemia

Hyperlipidemia is an elevation of lipids in the bloodstream. These lipids include, for example, cholesterol and triglycerides. General hyperlipidemia is determined by the results of a lipid profile. The lipid profile includes LDL, HDL, triglycerides and total cholesterol measurements. A group of persons having hyperlipidemia with a total cholesterol level greater than 240 mg/dl, an HDL (high density lipid) of below 40 mg/ml, a triglyceride level of greater than 200 mg/dl and an LDL (low density lipid) level of over 160 mg/ml, after a 9 to 12 hours of fasting, are chosen for this study.

10 people are given a treatment of ritonavir at 600 mg taken twice a day. The other 10 people are given a placebo. The treatment continued for 2 months.

The study group is advised to follow their normal routine concerning their eating habits and exercise patterns, which is recorded 1 month prior to this study and throughout this study.

At the end of two months another fasting (9-12 hours) lipid profile is taken. The people given ritonavir have significantly reduced levels of total cholesterol, triglycerides and LDL and higher levels of HDL than those in the control group.

### Example 9

### Treating Hyperlipidemia

The same study in Example 8 is conducted with a larger group of people having hyperlipidemia. Each of the treated groups 1 to 7 is given as amprenavir, atazanavir, indinavir, lopinavir, fosamprenavir, nelfinavir or saquinavir in the same amounts as set forth in Example 3. The control group is given a placebo. At the end of two months another fasting (9-12 hours) lipid profile is taken. The people given amprenavir, atazanavir, indinavir, lopinavir, fosamprenavir, nelfinavir or saquinavir have significantly reduced levels of cholesterol, triglycerides and LDL and higher levels of HDL than those in the control group.

1. A method of reducing body fat content of a subject in need thereof, the method comprising administering to the subject a therapeutically effective amount of an agent capable of down-regulating activity and/or expression of at least one component participating in protein digestion and/or absorption.

2. The method of claim 1, wherein said component participating in protein digestion and/or absorption is a protease.

3. The method of claim 2, wherein said protease is at least one component of an enteropeptidase pathway.

4. The method of claim 3, wherein said at least one component of an enteropeptidase pathway is a serine-protease.

5. The method of claim 3, wherein said at least one component of an enteropeptidase pathway is an activator of enteropeptidase.

6. The method of claim 4, wherein said at least one component of an enteropeptidase pathway is enteropeptidase.

7. The method of claim 3, wherein said at least one component of an enteropeptidase pathway is a downstream effector of enteropeptidase.

8. The method of claim 7, wherein said downstream effector of enteropeptidase is trypsin.

9. The method of claim 2, wherein said protease is an aspartate-protease.

10. The method of claim 9, wherein said protease is a pepsin.

11. The method of claim 10, wherein said pepsin is selected from the group consisting of Pepsin A, Pepsin B and Gastricin.

12. The method of claim 1, wherein down-regulating activity and/or expression of at least one component participating in protein digestion and/or absorption is effected by an agent selected from the group consisting of:
(i) an oligonucleotide directed to an endogenous nucleic acid sequence expressing said at least one component participating in said protein digestion and/or absorption;
(ii) a protease inhibitor directed to said at least one component participating in protein digestion and/or absorption

13. The method of claim 12, wherein said protease inhibitor is an aspartic protease inhibitor.

14. The method of claim 13, wherein said aspartic protease inhibitor is a peptidomimetic aspartic protease inhibitor.

15. The method of claim 13, wherein said aspartic protease inhibitor is a low molecular weight aspartic protease inhibitor.

16. The method of claim 15, wherein said low molecular weight aspartic protease inhibitor is pepstatin.

17. The method of claim 13, wherein said aspartic protease inhibitor is extracted from a plant.

18. The method of claim 17, wherein said plant is selected from the group consisting of Solanum tuberosum (potato), Cucurbita maxima (squash) and Anchusa strigosa (Prickly Alkanet).

19. The method of claim 13, wherein said aspartic protease inhibitor is extracted from a parasite.

20. The method of claim 19, wherein said parasite is selected from the group Ascaris suum and Ascaris lombricoides.

21. The method of claim 19, wherein said aspartic protease inhibitor is PI-3.

22. The method of claim 12, wherein said protease inhibitor is a serine protease inhibitor.

23. The method of claim 22, wherein said protease inhibitor is an inhibitor of enteropeptidase.

24. The method of claim 22, wherein said protease inhibitor is an inhibitor of trypsin.

25. The method of claim 12 wherein said oligonucleotide is DNA or RNA.

26. The method of claim 25 wherein said oligonucleotide is complementary to SEQ ID NO:1 or homologues thereof.

27. The method of claim 25 wherein said oligonucleotide is complementary to SEQ ID NO:2 or homologues thereof.

28. The method of claim 22, wherein said serine protease inhibitor is a low molecular weight serine protease inhibitor.

29. The method of claim 22, wherein said serine protease inhibitor is a peptidomimetic serine protease inhibitor.

30. The method of claim 22 wherein said serine protease inhibitor is an inhibitor of both enteropeptidase and trypsin.

31. The method of claim 1, wherein said agent is linked to a mucoadhesive agent.

32. The method of claim 31, wherein said mucoadhesive agent is a mucoadhesive polymer.

33. The method of claim 32, wherein said mucoadhesive polymer is selected from the group consisting of chitosan, polyacrylic acid, hydroxyprpyl methylcellulose and hyaluronic acid.

34. The method of claim 1, wherein said subject in need thereof is afflicted with a condition or disorder selected from the group consisting of excessive weight, obesity, type II diabetes, hypercholesterolemia, atherosclerosis, hypertension, pancreatitis, hypertriglyceridemia and hyperlipidemia, or is a non-diabetic or non-pancreatitis patient.

35. The method of claim 1, wherein said administering to the subject is effected by oral administration.


SEQUENCE LISTING (10 sequences)

SEQ ID NO: 1 - DNA, 800 bp, artificial sequence
  Site of cleavage by enteropeptidase
  CDS (7)..(750)
accaccatgaatccactcctgatccttacctttgtggcagctgctcttgctgccccctttgatgatgatgacaagatcgttgggggctacaactgtgaggagaattctgtcccctaccaggtgtccctgaattctggctaccacttctgtggtggctccctcatcaacgaacagtgggtggtatcagcaggccactgctacaagtcccgcatccaggtgagactgggagagcacaacatcgaagtcctggaggggaatgagcagttcatcaatgcagccaagatcatccgccacccccaatacgacaggaagactctgaacaatgacatcatgttaatcaagctctcctcacgtgcagtaatcaacgcccgcgtgtccaccatctctctgcccaccgcccctccagccactggcacgaagtgcctcatctctggctggggcaacactgcgagctctggcgccgactacccagacgagctgcagtgcctggacgctcctgtgctgagccaggctaagtgtgaagcctcctaccctggaaagattaccagcaacatgttctgtgtgggcttccttgagggaggcaaggattcatgtcagggtgattctggtggccctgtggtctgcaatggacagctccaaggagttgtctcctggggtgatggctgtgcccagaagaacaagcctggagtctacaccaaggtctacaactatgtgaaatggattaagaacaccatagctgccaatagctaaagcccccagtatctcttcagtctctataccaataaagtgaccctgttctc

SEQ ID NO: 2 - protein, 247 aa, artificial sequence
  Site of cleavage by enteropeptidase
MNPLLILTFVAAALAAPFDDDDKIVGGYNCEENSVPYQVSLNSGYHFCGGSLINEQWVVSAGHCYKSRIQVRLGEHNIEVLEGNEQFINAAKIIRHPQYDRKTLNNDIMLIKLSSRAVINARVSTISLPTAPPATGTKCLISGWGNTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 3 - DNA, 3696 bp, Homo sapiens
  CDS (41)..(3100)
accagacagttcttaaattagcaagccttcaaaaccaaaaatggggtcgaaaagaggcatatcttctaggcatcattctctcagctcctatgaaatcatgtttgcagctctctttgccatattggtagtgctctgtgctggattaattgcagtatcctgcctgacaatcaaggaatcccaacgaggtgcagcacttggacagagtcatgaagccagagcgacatttaaaataacatccggagttacatataatcctaatttgcaagacaaactctcagtggatttcaaagttcttgcttttgaccttcagcaaatgatagatgagatctttctatcaagcaatctgaagaatgaatataagaactcaagagttttacaatttgaaaatggcagcattatagtcgtatttgaccttttctttgcccagtgggtgtcagatcaaaatgtaaaagaagaactgattcaaggccttgaagcaaataaatccagccaactggtcactttccatattgatttgaacagcgttgatatcctagacaagctaacaaccaccagtcatctggcaactccaggaaatgtctcaatagagtgcctgcctggttcaagtccttgtactgatgctctaacgtgtataaaagctgatttattttgtgatggagaagtaaactgtccagatggttctgacgaagacaataaaatgtgtgccacagtttgtgatggaagatttttgttaactggatcatctgggtctttccaggctactcattatccaaaaccttctgaaacaagtgttgtctgccagtggatcatacgtgtaaaccaaggactttccattaaactgagcttcgatgattttaatacatattatacagatatattagatatttatgaaggtgtaggatcaagcaagattttaagagcttctatttgggaaactaatcctggcacaataagaattttttccaaccaagttactgccacctttcttatagaatctgatgaaagtgattatgttggctttaatgcaacatatactgcatttaacagcagtgagcttaataattatgagaaaattaattgtaactttgaggatggcttttgtttctgggtccaggatctaaatgatgataatgaatgggaaaggattcagggaagcaccttttctccttttactggacccaattttgaccacacttttggcaatgcttcaggattttacatttctaccccaactggaccaggagggagacaagaacgagtggggcttttaagcctccctttggaccccactttggagccagcttgccttagtttctggtatcatatgtatggtgaaaatgtccataaattaagcattaatatcagcaatgaccaaaatatggagaagacagttttccaaaaggaaggaaattatggagacaattggaattatggacaagtaaccctaaatgaaacagttaaatttaaggttgcttttaatgcttttaaaaacaagatcctgagtgatattgcgttggatgacattagcctaacatatgggatttgcaatgggagtctttatccagaaccaactttggtgccaactcctccaccagaacttcctacggactgtggaggaccttttgagctgtgggagccaaatacaacattcagttctacgaactttccaaacagctaccctaatctggctttctgtgtttggattttaaatgcacaaaaaggaaagaatatacaacttcattttcaagaatttgacttagaaaatattaacgatgtagttgaaataagagatggtgaagaagctgattccttgctcttagctgtgtacacagggcctggcccagtaaaggatgtgttctctaccaccaacagaatgactgtgcttctcatcactaacgatgtgttggcaagaggagggtttaaagcaaactttactactggctatcacttggggattccagagccatgcaaggcagaccattttcaatgtaaaaatggagagtgtgttccactggtgaatctctgtgacggtcatctgcactgtgaggatggctcagatgaagcagattgtgtgcgttttttcaatggcacaacgaacaacaatggtttagtgcggttcagaatccagagcatatggcatacagcttgtgctgagaactggaccacccagatttcaaatgatgtttgtcaactgctgggactagggagtggaaactcatcaaagccaatcttctctaccgatggtggaccatttgtcaaattaaacacagcacctgatggccacttaatactaacacccagtcaacagtgtttacaggattccttgattcggttacagtgtaaccataaatcttgtggaaaaaaactggcagctcaagacatcaccccaaagattgttggaggaagtaatgccaaagaaggggcctggccctgggttgtgggtctgtattatggcggccgactgctctgcggcgcatctctcgtcagcagtgactggctggtgtccgccgcacactgcgtgtatgggagaaacttagagccatccaagtggacagcaatcctaggcctgcatatgaaatcaaatctgacctctcctcaaacagtccctcgattaatagatgaaattgtcataaaccctcattacaataggcgaagaaaggacaacgacattgccatgatgcatctggaatttaaagtgaattacacagattacatacaacctatttgtttaccggaagaaaatcaagtttttcctccaggaagaaattgttctattgctggttgggggacggttgtatatcaaggtactactgcaaacatattgcaagaagctgatgttcctcttctatcaaatgagagatgccaacagcagatgccagaatataacattactgaaaatatgatatgtgcaggctatgaagaaggaggaatagattcttgtcagggggattcaggaggaccattaatgtgccaagaaaacaacaggtggttccttgctggtgtgacctcatttggatacaagtgtgccctgcctaatcgccccggagtgtatgccagggtctcaaggtttaccgaatggatacaaagttttctacattagcgcatttcttaaactaaacaggaaagtcgcattattttcccattctactctagaaagcatggaaattaagtgtttcgtacaaaaattttaaaaagttaccaaaggtttttattcttacctatgtcaatgaaatgctagggggccagggaaacaaaattttaaaaataataaaattcaccatagcaatacagaataactttaaaataccattaaatacatttgtatttcattgtgaacaggtatttcttcacagatctcatttttaaaattcttaatgattatttttattacttactgttgtttaaagggatgttattttaaagcatataccatacacttaagaaatttgagcagaatttaaaaaagaaagaaaataaattgtttttcccaaagtatgtcactgttggaaataaactgccataaattttctagttccagtttagtttgctgctattagcagaaactcaattgtttctctgtcttttctatcaaaattttcaacatatgcataaccttagtattttcccaaccaatagaaactatttattgtaagcttatgtcacaggcctggactaaattgattttacgttcctctt

SEQ ID NO: 4 - protein, 1019 aa, Homo sapiens
MGSKRGISSRHHSLSSYEIMFAALFAILVVLCAGLIAVSCLTIKESQRGAALGQSHEARATFKITSGVTYNPNLQDKLSVDFKVLAFDLQQMIDEIFLSSNLKNEYKNSRVLQFENGSIIVVFDLFFAQWVSDQNVKEELIQGLEANKSSQLVTFHIDLNSVDILDKLTTTSHLATPGNVSIECLPGSSPCTDALTCIKADLFCDGEVNCPDGSDEDNKMCATVCDGRFLLTGSSGSFQATHYPKPSETSVVCQWIIRVNQGLSIKLSFDDFNTYYTDILDIYEGVGSSKILRASIWETNPGTIRIFSNQVTATFLIESDESDYVGFNATYTAFNSSELNNYEKINCNFEDGFCFWVQDLNDDNEWERIQGSTFSPFTGPNFDHTFGNASGFYISTPTGPGGRQERVGLLSLPLDPTLEPACLSFWYHMYGENVHKLSINISNDQNMEKTVFQKEGNYGDNWNYGQVTLNETVKFKVAFNAFKNKILSDIALDDISLTYGICNGSLYPEPTLVPTPPPELPTDCGGPFELWEPNTTFSSTNFPNSYPNLAFCVWILNAQKGKNIQLHFQEFDLENINDVVEIRDGEEADSLLLAVYTGPGPVKDVFSTTNRMTVLLITNDVLARGGFKANFTTGYHLGIPEPCKADHFQCKNGECVPLVNLCDGHLHCEDGSDEADCVRFFNGTTNNNGLVRFRIQSIWHTACAENWTTQISNDVCQLLGLGSGNSSKPIFSTDGGPFVKLNTAPDGHLILTPSQQCLQDSLIRLQCNHKSCGKKLAAQDITPKIVGGSNAKEGAWPWVVGLYYGGRLLCGASLVSSDWLVSAAHCVYGRNLEPSKWTAILGLHMKSNLTSPQTVPRLIDEIVINPHYNRRRKDNDIAMMHLEFKVNYTDYIQPICLPEENQVFPPGRNCSIAGWGTVVYQGTTANILQEADVPLLSNERCQQQMPEYNITENMICAGYEEGGIDSCQGDSGGPLMCQENNRWFLAGVTSFGYKCALPNRPGVYARVSRFTEWIQSFLH

SEQ ID NO: 5 - DNA, 25 bp, artificial sequence
  Oligonucleotide ODN2053
  misc_feature: m 2′O-methyl modification in position 5′
cctgcctgggtgtcacttcmcmamc

SEQ ID NO: 6 - DNA, 26 bp, artificial sequence
  Oligonucleotide ODN2154
  misc_feature: m 2′O-methyl modification in position 5′
gcagcagacaccagccagtcmamamu

SEQ ID NO: 7 - DNA, 24 bp, artificial sequence
  Oligonucleotide ODN1160
  misc_feature: m 2′O-methyl modification in position 5′
gtaggatgctctggtggamgmgmg

SEQ ID NO: 8 - DNA, 26 bp, artificial sequence
  Oligonucleotide ODN2689
  misc_feature: m 2′O-methyl modification in position 5′
cccagggtgattaggcagtgmcmamc

SEQ ID NO: 9 - DNA, 25 bp, artificial sequence
  Oligonucleotide ODN1527
  misc_feature: m 2′O-methyl modification in position 5′
cctggcagggctgtggaatmcmcmc

SEQ ID NO: 10 - protein, 12 aa, artificial sequence
  Site of cleavage by enteropeptidase
APFDDDDKIVGG